SEQUENCE LISTING (4 sequences)

SEQ ID NO: 1 - DNA, 1728 bp, RAT
  CDS (1)..(1728)
atgggggcgatggcaccgcgcacgctgctcctgctgctggcggccgccctggccccgacccagacccgcgcgggctctcactcgatgcggtatttcgacatcgccgtgtcccggcccggcctcggggagccccggtacatctctgtcggctacgtggaccacacggagttcgtgcgcttcgacagcgacgcggagaatccgagatacgagccgcgggcgccgtggatggagcgggaggggccggactattgggagagggagacacagaaagccaagggaaacgagcagaattaccgagtgagcctgaggaatctgcgcggctactacaaccagagcgagggcggctctcacaccatccagaggatgtatggctgtgacgtggggacggacgggagcctcctccgcgggtataggcaggacgcctacgacggccgcgattacatcgccctgaacgaagacctgaagacgtggacggcggcggactttgcggcacagatcacccggaacaagtgggatcgggctggtgttgcagagagactcagggcctacctggagggcacgtgcgtggagtggctccgcagatacctggagcacgggaaggagacgctgctgcgcttagatcccccaaaggcacatgtgacccttcaccccagacctgaaggtgatgtcaccctgaggtgctgggccctgggcttctaccctgctgacatctccctgagctggcagttgaatggggaggacctgacccaggacatggagcttgtggagaccaggcctgcaggggatggaaccttccagaagtgggcatctgtggtggtgccacttgggaaggagcagaattacacatgccgtgtggagcatgaggggctgcctgagccgcttacccagagatgggagccttctctatccaccgactccaacatggaaacctatgtcatttatgtcgtccttggagctgtggccatcattgcagctgtgatcattgcagctgtggtggctgttgtgaggaggaggaggaagagaaacacagagcccagaagacccaagcccagaccccccacagatatctgttcatgtgatgacaacttgggtagaccatctgtcttcatcttccccccaaagcccaaggatatactcatgatcaccctgacccccaaggtcacctgtgtggtggtggatgtgagcgaggaggagccagacgtccagttcagctggtttgtggacaacgtacgagtattcacagctcagacacaaccccatgaggagcagctcaacggtaccttccgagtggtcagtaccctccatatccagcaccaggactggatgagcggcaaggagttcaaatgcaaggtcaacaacaaagacctcccaagccccatcgagaaaaccatctcaaaacccagaggaaaagcccggacacctcaagtatacaccattcctccacctcgtgaacaaatgtccaagaataaggttagcctcacctgcatggtcaccagcttctaccccgcatccatcagtgtggagtgggaaaggaatggggagctggagcaggactacaagaacaccctacccgtgctggactcagatgagtcctacttcctctacagcaagctcagtgtggacacggacagttggatgcgaggagacatttatacctgctctgtggtgcacgaggctcttcataaccaccacacacagaagaacctgtcccgctctcctggtaaatga

SEQ ID NO: 2 - protein, 575 aa, RAT
MGAMAPRTLLLLLAAALAPTQTRAGSHSMRYFDIAVSRPGLGEPRYISVGYVDHTEFVRFDSDAENPRYEPRAPWMEREGPDYWERETQKAKGNEQNYRVSLRNLRGYYNQSEGGSHTIQRMYGCDVGTDGSLLRGYRQDAYDGRDYIALNEDLKTWTAADFAAQITRNKWDRAGVAERLRAYLEGTCVEWLRRYLEHGKETLLRLDPPKAHVTLHPRPEGDVTLRCWALGFYPADISLSWQLNGEDLTQDMELVETRPAGDGTFQKWASVVVPLGKEQNYTCRVEHEGLPEPLTQRWEPSLSTDSNMETYVIYVVLGAVAIIAAVIIAAVVAVVRRRRKRNTEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 3 - DNA, 1593 bp, Homo sapiens
  CDS (1)..(1593)
atggccgtcatgccgccccgaaccctcctcctgctactctcgggggccctggccctgacccagacctgggcaggctcccactccatgaggtatttcttcacatccgtgtcccggcccggccgcggggagccccgcttcatcgcagtgggctacgtggacgactcgcagttcgtgcagttcgacagcgacgccgcgagccagaggatggagccgcgggcgccgtggatagagcaggaggagccggagtattgggacgaggagacacggaatgtgaaggcccactcacagactaaccgagcgaacctggggaccctgcgcggctactacaaccagagcgaggacggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccgtcgggcggagcagctgagagcctacctggagggcgagtgcgtggacgggctccgcagatacctggagaacgggaaggagacgctgcagcgcacggacccccccaagacacatatgacccaccaccccatctctgaccatgaggccactctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtaccttctggaaaggagaagagatacacctgccatgtgcagcatgagggtctgcccgagcccctcaccctgagatgggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 530 aa, Homo sapiens [sequence omitted]

(19) United States
(12) Patent Application Publication
(10) Pub. No.: US 2005/0287631 A1
(43) Pub. Date: Dec. 29, 2005

(54) Compositions and methods related to a dimeric MHC class I and II-Like molecule (dsMHCI and dsMHCII)

(76) Inventors: Martin Kroenke, Koln (DE); Nicholas Zavazava, Coralville, IA (US)

Correspondence Address: FULBRIGHT & JAWORSKI L.L.P., 600 CONGRESS AVE., SUITE 2400, AUSTIN, TX, 78701, US

(21) Appl. No.: 10/995,805

(22) Filed: Nov. 23, 2004

Related U.S. Application Data
(60) Provisional application No. 60/524,988, filed on Nov. 25, 2003.

(51) Int. Cl. A61K 38/17; C07K 14/74; C07H 21/04

(52) U.S. Cl. 435/69.1; 435/320.1; 435/325; 514/12; 530/350; 536/23.5

(57) ABSTRACT

Embodiments of the invention include dimeric soluble MHC molecules (dsMHCs) and compositions and methods for their use. The molecules may be used to downregulate activated T cells. The dsMHCs may be administered by intraperitoneal infusion into a tissue or an organ recipient. The administration of dsMHCs molecules may also be supplemented with immunosupressant to induce the engraftment of allografts. dsMHCs molecules may also be used for visualization of alloreactive T cells in various tissues or organs.

This application claims priority to U.S. Provisional Patent application Ser. No. 60/524,988, filed on Nov. 25, 2003 entitled “Compositions and Methods Related to a Dimeric MHC Class I and II-Like Molecule (dsMHCI and dsMCHII),” which is incorporated herein by reference in its entirety.

### BACKGROUND OF THE INVENTION

A. Field of the Invention

The present invention relates generally to the fields of medicine and immunology. More particularly, it concerns compositions and methods related to identification, modulation and/or abrogation of alloreactive T cells.

B. Description of Related Art

Membrane-bound major histocompatibility complex (MHC) molecules present antigenic and/or endogenous peptides to T cells, which leads to T cell stimulation if co-stimulation is provided (Gill et al., 1996; June et al., 1994). In transplantation, antigen presentation is unique because both donor-derived cells (so called “passenger leukocytes”) within the graft, as well as host-derived antigen presenting cells, present alloantigen to recipient T cells. This leads to direct and indirect alloantigen recognition, respectively (Shoskes and Wood, 1994).

Little is known about the immunomodulatory role of soluble MHC (sMHC) antigens shed into the circulation. The inventors and others have characterized the biochemistry of serum-derived sMHC and shown quantitative differences between individuals of different human leukocyte antigen (HLA) haplotypes (Haga et al., 1991; Kao et al., 1988; Zavazava et al., 1990; Pouletty et al., 1993). For example, HLA-A24 and HLA-B15 positive individuals constitutively express 3-4 times more sMHC than other individuals (Zavazava et al., 1990; Pouletty et al., 1993). During inflammation, however, such as organ rejection episodes, sMHC class I levels are highly elevated, presumably as a response to pro-inflammatory cytokines (Zavazava et al., 1993). These observations raise the possibility of an in vivo role of sMHCs as immunoregulatory agents.

Studies designed to demonstrate the interaction of T cells and sMHC have failed in most cases. For example, Priestley et al. (1989) used a bolus of donor-derived sMHC to promote graft survival, but failed to show any effect. Equally, sMHC failed to affect bulk cultures of alloreactive cytotoxic T cells. However, the inventors previously described inhibition of T cells in vitro by sMHC antigens purified by affinity chromatography (Zavazava et al., 1991; Hausmann et al., 1993). These data were in agreement with studies by others using alloreactive T cells (Schneck et al., 1989a; Schneck et al., 1989b). Physical blockage of the T cell receptor (TcR) could be ruled out as a mechanism for this inhibition based on the published Kₐ for TcR-ligand binding (Schodin et al., 1996) or the estimated K_{d} values for TcR/MHC or TcR/peptide interactions. The inventors extended these studies and demonstrated for the first time that sMHC induce apoptosis to alloreactive T cells (Zavazava and Kronke, 1996; Hansen et al., 1998). Similar data were obtained by others with sMHC class II molecules (Arimilli et al., 1996; Nag et al., 1996; O'Herrin et al., 2001).

In studies carried out by the inventors, it was demonstrated that apoptosis was due to upregulation of FasL (CD95L) and subsequent suicide killing. Also, apoptosis was highest in cells with a high affinity for alloantigen. The induction of apoptosis could be blocked by an anti-FasL antibody and could be prevented by the provision of co-stimulation. It has been suggested that sMHC-induced apoptosis may involve CD8 (Ghio et al., 2000).

Data on the use of sMHC antigens to modulate immune-responses in vivo have been rare. In preliminary studies, the inventors have shown that monomeric MHC class I prolong graft survival in about 50% of the transplanted allografts (Behrens et al., 2001). However, permanent engraftment required the addition of low dose CsA. Others have specifically inhibited rejection of skin grafts using a MHC class I binding domain fused to an IgG molecule that included the IgG variable region (Schneck et al., 1996). These data established that sMHC complexes are capable of suppressing T cell responses in vivo. Similarly, antigen-specific inhibition of an alloreactive TCR-transgenic T cell population in vivo resulted in consequent outgrowth of an allogeneic tumor (O'Herrin et al., 2001) indicating that MHC molecules are powerful immunomodulatory reagents. More recently, Casares et al. (2002) have demonstrated that a class II dimeric molecule prevented the onset of disease and restored normoglycemia to diabetic animals.

An advantage in advancing these studies is that they may eliminate the need for the use of toxic treatments in T-cell mediated diseases in general and may provide a more effective T-cell specific prophylactic or therapeutic therapy.

### SUMMARY OF THE INVENTION

Embodiments of the invention include a polypeptide comprising MHC class I alpha 1, alpha 2 and alpha 3 domains or MHC class II extracellular domains operatively coupled to a dimerization domain, wherein the polypeptide lacks an immunoglobulin variable region domain. The dimerization domain can be an immunoglobulin C2-C3 domain or any suitable soluble protein that can be used as a scaffold, which may also include an immunoglobulin hinge region or other peptide spacer. The polypeptide may further comprise an amino terminal leader sequence. The leader sequence can be a MHC class I or MHC class II leader sequence, or other leader sequence known in the art, used for production of the polypeptide in a host cell or organism. The polypeptide can further comprise a carboxy-terminal peptide tag, for example a Flag tag.

Certain embodiments of the invention include a polynucleotide comprising a nucleic acid sequence that encodes a polypeptide comprising MHC class I alpha 1, alpha 2 and alpha 3 domains or MHC class II extracellular domains fused with a carboxy-terminal dimerization domain, wherein the polypeptide lacks an immunoglobulin variable region. The polynucleotide can further be comprised in an expression cassette comprising a promoter, which may then be comprised in an expression vector. The expression vector is typically a eukaryotic expression vector, such as a plasmid or viral expression vector. The polynucleotide may encode a fusion protein with a carboxy-terminal peptide tag and/or an amino terminal leader sequence.

Various embodiments of the invention include a proteinaceous composition. A proteinaceous composition will typically comprise a dimeric or homodimeric molecule, wherein each monomer comprises MHC class I alpha 1, alpha 2 and alpha 3 domains or MHC class II extracellular domains operatively coupled to a dimerization domain, wherein each monomer lacks an immunoglobulin variable domain. The dimerization domain can be an immunoglobulin C2-C3 domain. The dimerization domain may also include an immunoglobulin hinge region or a similar peptide spacer region between the MHC binding domain and a dimerization domain. Each monomer will typically comprise an amino terminal leader sequence. The leader sequence, for example may be an MHC class I leader sequence. In some embodiments, at least one monomer can comprise a carboxy-terminal peptide tag, for example a Flag tag. A proteinaceous composition of the invention is typically a pharmaceutically acceptable composition.

Embodiments of the invention include proteinaceous compositions comprising a dimeric complex of polypeptides, wherein a first and second polypeptide comprise MHC I or MHC II antigen binding region operatively coupled to at least one immunoglobulin constant region, wherein the first and second polypeptides each lack immunoglobulin variable regions.

Other embodiments include a cell comprising a nucleic acid coding for a polypeptide comprising MHC I or MHC II antigen binding region operatively coupled to a dimerization domain, wherein the polypeptide lacks an immunoglobulin variable region domains.

Certain embodiments include a method of producing a dimeric polypeptide molecule comprising contacting a host cell with a nucleic acid encoding a polypeptide comprising MHC class I alpha 1, alpha 2 and alpha 3 domains or MHC class II extracellular domains operatively coupled to a dimerization domain, wherein the polypeptide lacks an immunoglobulin variable region domains; and isolating a dimeric polypeptide molecule.

Various embodiments of the invention include a method comprising contacting an alloreactive T-cell with an effective amount of a dimeric molecule comprising a first and second polypeptide comprising MHC I or MHC II antigen binding region operatively coupled to at least one immunoglobulin constant region, wherein the first and second polypeptides each lack an immunoglobulin variable region. The T-cell may or may not be in a subject. The T-cell is typically in or derived from a tissue transplant or a sample from a tissue transplant, respectively. The tissue may be all or part of an organ or organ system. The methods of the invention may further comprise administering to the subject an effective dose of an immunosuppressant or other therapeutic molecule or medicament. The immunosuppressant can be cyclosporin A. An effective amount of the dimeric molecule can be at a dose of about 1, 2, 3, 4, 5, 6, 7, 8, 9, 10, 11, 12, 13, 14, 15, 20, 25, 30, 35, 40, or 45 μg to 50, 55, 60, 65, 70, 75, 80, 85, 90, 95, or 100 μg per kg of body weight. In certain embodiments the dose is typically about 10 μg to 50 μg per kg of body weight. The dimeric molecule may be administered by intralymphatic, intraossicular, intravascular, intravenous, peritoneal or intraperitoneal injection, perfusion and/or infusion.

Embodiments of the invention may include a method comprising contacting a sample suspected of containing alloreactive T-cells with an effective amount of a dimeric molecule comprising a first and second polypeptide comprising MHC I or MHC II antigen binding region operatively coupled to at least one immunoglobulin constant region, wherein the first and second polypeptides each lack an immunoglobulin variable region; and visualizing a T-cell by contacting the T-cell/dimeric molecule complex with a detection reagent. A sample includes, but is not limited to peripheral blood, splenocytes, or all or part of a tissue or organ.

Certain embodiments of the invention include a kit comprising a first container comprising a proteinaceous composition including a dimeric complex of polypeptides, wherein a first and second polypeptide comprise a MHC I or MHC II antigen binding region operatively coupled to at least one immunoglobulin constant region, wherein the first and second polypeptides each lack an immunoglobulin variable region. The kit can further comprise a second container comprising a reagent for detection or combination treatment with the dimeric polypeptide.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Schematic structure and characterization of the dimeric RT1.A1-Fc. (FIG. 1A) Schematic diagram of the dimer which was made up of a Flag tag, the Fc region of the rat IgG2c fragment fused through the hinge region to the MHC class I molecule. (FIG. 1B) The dimer was precipitated with the OX-18 MoAb (anti-RT1.A heavy chain, lane 1) and an anti-β2 microglobulin polyclonal serum (lane 6), respectively. Lanes 2 and 5 represent the vector controls and Lanes 3 and 4 represent the precipitates with the respective IgG isotype controls. (FIG. 1C) To determine the molecular size of the dimmer, gel filtration of the supernatant was performed. Two main peaks of 66 kD and 200 kD, respectively, were observed. The dimeric MHC molecule used in these experiments was detected in the 200 kD peak, confirming that the molecule was indeed dimeric and had the expected molecular size. (FIG. 1D) A single band of approximately 67 kD representing the single RT1.A1-Fc single chain was observed in the purified sample (lane 1). Lanes 2 and 3 represent the control supernatants of the wild type cells and that of Lewis-derived splenocytes, respectively.

FIGS. 2A-2C. Dimeric sMHC class I antigens induce permanent engraftment of donor-derived cardiac allografts. (FIG. 2A) DA recipient rats were transplanted Lewis-derived cardiac allografts and simultaneously treated with a single dose of 10 μg of dimeric RT1.A1-Fc daily for 14 days. Animals that received an additional dose of cyclosporin A permanently accepted cardiac allografts, n=12. Third-party allografts were acutely rejected. Both the dimer and CsA used alone moderately prolonged graft survival. (FIGS. 2B-2C) Histological sections revealed normal architecture of tolerated allografts 150 days post-transplantation, whereas rejected allografts were heavily infiltrated by mononuclear cells showing significant architectural damage to the allografts.

FIGS. 3A-3G. Treatment of recipient rats with dimeric MHC antigens abrogates T cell proliferation and cytotoxic T cell cytotoxicity. (FIG. 3A) To test the effect of dimer treatment on T cell response to alloantigen presented by direct presentation, CD8+ T cells from animals treated either with donor splenocytes only, donor splenocytes and the dimer, the dimer only or control untreated animals were used in a 5-day mixed lymphocyte reaction assay as responder cells. Irradiated Lewis-derived splenocytes were used as stimulator cells. Treatment with dimeric MHC strongly abrogated the alloproliferative response of T cells in animals that were sensitized with donor splenocytes and the dimer, compared to T cells in animals sensitized with splenocytes only, p<0,05, n=5. Third-party BN-derived stimulator cells remained unchanged in all groups. (FIG. 3B) CD8+ T cells were used as effector cells in a 4 h 51chromium release assay after restimulation of the cells from the various animal groups for 5 days in vitro using Lewis-derived splenocytes as stimulator cells. T cells derived from animals sensitized with splenocytes only showed strong target cell killing, whereas the cytotoxicity of cells recovered from animals treated with both splenocytes and the dimer was drastically abrogated, indicating a direct influence of the dimer. As expected, CD8+ T cells derived from control animals or from animals treated with the dimer only showed low cytotoxicity. (FIG. 3C) To determine whether the soluble dimer directly abrogates cytotoxic T cells, DA anti-Lewis, DA anti-BN or Lewis anti-DA CTL were incubated with the dimer and their cytotoxicity against appropriate Con A target cells tested in a 4 h-51chromium release assay. Anti-Lewis CTL were inhibited in a concentration-dependent manner, but not the DA anti-BN or the Lewis anti-DA CTL used as controls. (FIG. 3D) To determine the effect of the dimer treatment on CD4+ T cells, an ELISPOT was used for measuring the number of IFN-γ-secreting CD4+ T cells. Clearly dimer-treatment led to significant reduction in IFN-γ secretion by the CD4+ T cells. (FIG. 3E) To determine the ability of peritoneal macrophages to present the soluble dimeric MHC molecules, APC derived by adherence separation from splenocytes, lymphnodes or peritoneal macrophages of DA recipient animals were used to present the soluble dimer to DA-derived CD4+ T cells. APC derived from peritoneal macrophages failed to stimulate T cells. (FIGS. 3F-3G) To determine whether peritoneal macrophages traffic into the circulation after picking up alloantigen in the peritoneum, DA-derived peritoneal macrophages were pulsed with the dimer for 24 h ex vivo and re-infused into the same DA animals after labeling with CFSE. After another 24 h, the animals were sacrificed and green fluorescent cells detected in the peritoneum (FIG. 3F) and peripheral blood (FIG. 3G).

FIGS. 4A-4F. Dimeric RT1.A1-Fc visualizes intragraft and circulating alloreactive T cells. Immunohistochemical sections were incubated with the dimeric MHC molecule and binding visualized by alkaline phosphatase staining. Low staining of peripheral blood lymphocytes of tolerated allografts (FIG. 4A, ×200) and strong staining in rejected allografts (FIG. 4B, ×200) were observed. Interestingly, in some sections from tolerated allografts, such as in FIG. 2A, the inventors observed perivascular infiltrates, that however, were not accompanied by any pathology since the tissue was well maintained. These cells were predominantly CD4+. Control allografts from non-transplanted animals were negative for dimer staining (FIG. 4C, ×200). In peripheral blood of animals that rejected allografts, there were more dimer-staining CD8+ T cells (FIG. 4D), than in tolerated allografts (FIG. 4E) and even less in peripheral blood of animals transplanted with third-party BN allografts (FIG. 4F).

FIGS. 5A-5E. Acutely rejecting animals show a high frequency of dimer-binding cells in the spleen. Splenocytes of acutely rejecting (FIG. 5A) and those of control animals (FIG. 5B-5D) were separated by immunomagnetic beads and stained with the dimer. Cell fluorescence of dimer binding cells was highest in acutely rejecting animals and significantly lower in spleens of tolerated allografts and in the syngeneic (DA to Lewis) or third-party (BN to DA) graft controls. Pre-incubation of isolated CD8+ T cells from rejecting animals (FIG. 5A) with anti-FcR, -CD4, -CD8 or with anti-CD28 antibodies, respectively, had no significant effect on dimer binding by the CD8+ T lymphocytes as shown in the overlays (FIG. 5E).

### DESCRIPTION OF ILLUSTRATIVE EMBODIMENTS

In addressing the short comings of the art, in particular the need for a more efficient and effective method of detecting or evaluating alloreactivity of cells, tissues and organs, embodiments of the invention include engineered dimeric soluble MHC class I-like molecules (dsMHC I) or engineered dimeric soluble MHC class II-like molecules (dsMHC II), which lack immunoglobulin (Ig) variable regions and methods for their use in methods for diagnosis and detection. The lack of Ig variable regions reduce the immunogenicity of the molecule, as well as providing a less complex nucleic acid and protein to further enhance the isolation and therapeutic efficacy of the molecule. Molecules of the invention can be used to contact, bind, associate and/or interact with a T cell to regulate and/or identify an T cell or alloreactive T cell in vivo. In certain embodiments, dsMHC I or dsMHC II molecules may be used to prolong the presence of a donor cell, tissue, or organ in a recipient subject.

Molecules of the invention are engineered to provide MHC I or MHC II binding domain (binding domain) operatively linked to a dimerization moiety (see SEQ ID NO:1 or SEQ ID NO:3 for an exemplary nucleic acid sequence and see SEQ ID NO:2 or SEQ ID NO:4 for exemplary amino acid sequences). The MHC I binding domain typically will comprise alpha 1, alpha 2, alpha 3 domains or a combination thereof, of an MHC I molecule. The MHC II binding domain typically will comprise the extracellular domains of an MHC II molecule. Typically, the binding domain will be loaded with a peptide, preferably an allogeneic peptide, that is relevant to the identification or regulation of a T cell or all or part of a particular T cell population. Loading of a dsMHC I or dsMHC II (dsMHC or dsMHCs refers to dsMHC I and/or MHC II molecules) is typically accomplished by endogenous loading, that is peptides become associate the ligand binding domains of dsMHCs during transit of the protein(s) through the intracellular pathways that lead to secretion of dsMHCs. For example, a dsMHC I may be endogenously loaded by expression of a nucleic acid encoding the dsMHC I in a producer cell. A producer cell is a cell that is used to express dsMHCs. A culture or supernatant of a producer cell may be used as a starting material for purification of dsMHCs molecules. In one embodiment, the producer cell can be derived from a donor or a source of a transplant, or a cell, tissue or organ that is compatible with or having a similar genetic background or histocompatibility to that of the transplant. The producer cell does not need to be genetically similar to the donor since the nucleic acid encoding the dsMHC is donor type, thus a producer cell may be derived from a variety of cells in which the dsMHC is expressed. The source of the producer cell will at least be one that will allow the dsMHCs produced to identify or regulate a target T cell or T cell population. Typically, a producer cell will be used in vitro to produce the dsMHC. In vivo will typically entail engineering the molecule into recipient cells such as bone marrow cells, hepatocytes or other autologous or heterologous cells that could endogenously produce the dsMHC. The term “allogeneic” is used to refer a molecule, cell, tissue or organ that is derived from a genetically different source, although the source belongs to or is obtained from another member of the recipient species. In some embodiments a donor cell or tissue may be derived from a different species, i.e., a xenotransplant or xenogeneic donor.

A dimerization moiety may include portions of a immunoglobulin constant region. Immunoglobulin(s) or Ig(s) are a group of proteins that are products of antibody secreting cells. Igs are constructed of one, or several, units, each of which consists of two heavy (H) polypeptide chains and two light (L) polypeptide chains. Each unit possesses two combining sites for antigen, the variable regions. The H and L chains are made up of a series of domains. The H chains of Ig molecules are of several types, including μΔ, and γ (of which there are several subclasses), β and ∈. There are eight genetically and structurally identified Ig classes and subclasses as defined by heavy chain isotypes: IgM, IgD, IgG3, IgG1, IgG2b, IgG2a, IgE, and IgA. “IgG” means an immunoglobulin of the G class, and that, “IgG1” refers to an IgG molecules of subclass 1 of the G class. “Fab” and “F(ab′)₂” are fragments of Ig molecules that can be produced by proteolytic digestion of an intact Ig molecule. Digestion of an IgG molecule with papain will produce two Fab fragments and an Fc fragment and digestion with pepsin will produce an F(ab′)₂ fragment and subfragments of the Fc portion.

Typically, the heavy chain comprises a hinge region and constant regions (C). In particular embodiments constant regions C2 and C3 of an IgG molecule are used as a dimerization domain.

The dsMHCs of the invention may abrogate target cell lysis by CD8+ T cells, T cell ability to respond to non-self antigen or an alloantigen and/or reduce IFN-γ production by splenic CD4+ T cells. In various embodiments, the dsMHCs molecule of the invention may be used to induce or provide for graft or transplant tolerance. The dsMHCs may also be utilized in the visualization of reactive or alloreactive T cells in peripheral blood, splenocytes and explanted grafts or allografts revealing low frequency of reactive or alloreactive CD8+ T cells.

The inventors have exemplified certain embodiments of the invention by producing a dsMHC I of the Lewis rat strain and have shown in vivo, when infused intraperitoneally, that it induces graft tolerance to donor type cardiac allografts in DA recipients. Further, dsMHC I induced permanent engraftment of cardiac allografts was characterized by a very low presence of mononuclear cells within the parenchymal tissue of the graft. Ex vivo studies of splenocytes derived from tolerant animals showed low reactivity towards donor splenocytes in a 4-day mixed lymphocyte reaction. Third-party T cell reactivity was normal indicating that sMHC treatment did not lead to general immunosuppression, but rather antigen specific non-reactivity had been established. The dsMHC I can be used to visualize alloreactive T cells in peripheral blood, immunohisochemical sections and in splenocytes.

I. Histocompatibility Molecules

“Major Histocompatibility Complex” or “MHC” is a cluster of genes that plays a role in control of the cellular interactions responsible for physiologic immune responses. In mice, MHC antigens are called H-2 antigens (Histocompatibility-2 antigens). In humans, the MHC complex is known as the human leukocyte antigen (HLA) complex. For a detailed description of the MHC and HLA complexes, for review see Paul, 1993. Histocompatibility molecules are glycoproteins that are expressed on the surface of a cell. These molecules are responsible for the determination of self and non-self tissues or cells. There are two categories of MHC molecules class I (MHC I) and class II (MHC II). MHC I molecules contain a transmembrane molecule or heavy chain that comprises extracellular segments of α-helix that form an antigen binding groove, i.e. a binding domain or an antigen binding domain. Typically, a small peptide is bound in the groove formed by α-helices of the heavy chain. A beta-2 microglobulin (β₂M) molecule is non-covalently associated with the MHC I heavy chain. Humans produce three types of MHC I molecules, an HLA-A, HLA-B and HLA-C, that differ in composition of their heavy chain. MHC class II molecules are integral membrane glycoproteins consisting of a heavy chain with a molecular weight of approximately 34 kDa and a lighter chain with a molecular weight of approximately 29 kDa (Springer, 1977). The class II molecules consist of two domains (β1 and β1) forming the peptide-binding region (PBR) and two other immunoglobulin like domains (α2 and β2) forming the membrane proximal region. Human produce three types of MHC II molecules, HLA-DR, HLA-DQ, and HLA-DP.

T cells respond to antigens in the context of either Class I or Class II MHC molecules. Cytotoxic T cells respond mainly against foreign antigens in the context of Class I glycoproteins, such as viral-infected cells, tumor antigens and transplantation antigens. In contrast, helper T cells respond mainly against foreign antigens in the context of Class II molecules. Both types of MHC molecules are structurally distinct, but fold into very similar shapes. Each MHC molecule has a deep groove into which a short peptide, or protein fragment, can bind. Because this peptide is not part of the MHC molecule itself, it varies from one MHC molecule to the next. It is the presence of foreign peptides displayed in the MHC groove that engages clonotypic T cell receptors on individual T cells, causing them to respond to foreign antigens.

Antigen-specific recognition by T cells is based on the ability of clonotypic T cell receptor to discriminate between various antigenic-peptides resident in MHC molecules. These receptors have a dual specificity for both antigen and MHC (Zinkemagel et al., 1974). Thus, T cells are both antigen-specific and MHC-restricted. A simple molecular interpretation of MHC-restricted recognition by T cells is that TcRs recognize MHC residues as well as peptide residues in the MHC-peptide complex. Independent of the exact mechanism of recognition, the clonotypic T cell receptor is the molecule that is both necessary and sufficient to discriminate between the multitude of peptides resident in MHC.

T cells can be divided into two broad subsets; those expressing α/β TcR and a second set that expresses γ/Δ TcR. Cells expressing α/β TcR have been extensively studied and are known to comprise most of the antigen-specific T cells that can recognize antigenic peptide/MHC complexes encountered in viral infections, autoimmune responses, allograft rejection and tumor-specific immune responses. Cells expressing α/β TcRs can be further divided into cells that express CD8 accessory molecules and cells that express CD4 accessory molecules. While there is no intrinsic difference between the clonotypic α/β T cell receptors expressed either on CD4 and CD8 positive cells, the accessory molecules largely correlate with the ability of T cells to respond to different classes of MHC molecules. Class I MHC molecules are recognized by CD8+, or cytotoxic T cells and class II MHC molecules by CD4+, or helper T cells. There is a large degree of homology between both α/β and γ/Δ TcR expressed in rodents and humans. This extensive homology has, in general, permitted one to develop murine experimental models from which results and implications may be extrapolated to the relevant human counterpart. Certain embodiments of the invention are designed to target the CD8 T cell response.

A. The Role of MHC Molecules in Transplantation

MHC molecules play an essential role in determining the fate of grafts or transplants. Various species display major immunological functional properties associated with the MHC including, but not limited to, vigorous rejection of tissue grafts, stimulation of antibody production, stimulation of the mixed lymphocyte reaction (MLR), graft-versus-host reactions (GVH), cell-mediated lympholysis (CML), immune response genes, and restriction of immune responses. Transplant rejection occurs when skin, organs, or other tissues are transplanted across an MHC incompatibility. Graft rejection occurs when the immune system is activated by mismatched transplantation antigens that are present in donor tissue but not in recipient. Graft rejection may occur in the graft itself by exposure of circulating immune cells to foreign antigens, or it may occur in draining lymph nodes due to the accumulation of trapped transplantation antigens or graft cells. Because of the diversity of MHC antigens, numerous specificities are possible during physiological and pathophysiologic immune-related activities (e.g., transplantation, viral infections and tumor development). Recognized HLA specificities are depicted, for example, in a review by Bodmer et al. (1989).

B. Regulation of Immune Reponses

Interest in analyzing both normal and abnormal T cell-mediated immune responses led to the development of a series of novel soluble analogs of T cell receptors and MHC molecules to probe and regulate specific T cell responses. The development of these reagents was complicated by several facts. First, T cell receptors interact with peptide/MHC complexes with relatively low affinities (Matsui et al., 1991; Sykulev et al., 1994; Corr et al., 1994). In order to specifically regulate immune responses, soluble molecules with high affinities/avidities for either T cell receptors or peptide/MHC complexes are needed. However, simply making soluble monovalent analogs of either T cell receptors or peptide/MHC complexes has not proven to be effective at regulating immune responses with the required specificity and avidity.

To regulate immune responses selectively, investigators have made soluble versions of proteins involved in immune responses. Soluble divalent analogs of proteins involved in regulating immune responses with single transmembrane domains have been generated by several laboratories. Initially, CD4/Ig chimeras were generated (Capon et al., 1989; Bryn et al., 1990), as well as CR2/Ig chimeras (Hebell et al., 1991). Later it was demonstrated that immune responses could be modified using specific CTLA-4/Ig chimeras (Linsley et al., 1992; U.S. Pat. No. 5,434,131; Lenschow et al., 1992). In addition, class I MHC/Ig chimeras, which included the Ig variable region, were used to modify in vitro allogeneic responses (U.S. Pat. No. 6,458,354 and Dal Porto et al., 1993, each of which is incorporated herein by reference).

Embodiments of the present invention provide an improved dsMHC I or dsMHC II composition that comprises dimeric soluble molecules comprising a MHC binding domain and a dimerization domain, for example the hinge, C2, C3 region of an immunoglobulin. The dsMHC I or dsMHC II lacks an Ig variable region. The absence of the variable region provides a reduced immunogenicity and complex that provides for a more efficient and effective molecule.

II. Proteinaceous Compositions

In certain embodiments, a proteinaceous composition comprises at least one dsMHC I or dsMHC II molecule. The proteinaceous composition can comprise a biocompatible dsMHC I or dsMHC II molecule. As used herein, the term “biocompatible” refers to a substance which produces no significant untoward effects when applied to, or administered to, a given organism according to the methods and amounts described herein. Organisms include, but are not limited to, humans, mice, dogs, cats, livestock, domestic and wild animals. Untoward or undesirable effects are those such as significant toxicity or adverse immunological reactions. In preferred embodiments, biocompatible molecule containing compositions will generally be mammalian proteins or synthetic proteins each essentially free from toxins, pathogens and harmful immunogens.

The dsMHC I or ds MHC II polypeptide or molecule comprises at least one fusion protein, see SEQ ID NO:2 or SEQ ID NO:4 for exemplary amino acid sequences. The fusion protein comprises a dimerization domain, e.g., C2-C3 region of an immunoglobulin heavy chain (alternatively the hinge region) and an extracellular domain(s) of a MHC I or a MHC II polypeptide (antigen binding domain). The fusion proteins associate to form a dimeric soluble MHC I molecule (dsMHC I) a dimeric soluble MHC II molecule (dsMHC II). The dsMHC I comprises two antigen binding sites. Each antigen binding site is formed by the extracellular domains of the MHC I.

dsMHC I molecules of the invention can also be used to activate or inhibit alloreactive T cells. It is possible to conjugate toxin molecules, such as ricin or Pseudomonas toxin, to molecular complexes of the invention.

Proteinaceous compositions may be made by any technique known to those of skill in the art, including the expression of dsMHC I or dsMHC II polypeptides through standard molecular biological techniques or the chemical synthesis of proteinaceous materials. The nucleotide and polypeptide sequences for various MHC I, MHC II and immmoglobulin genes have been previously disclosed, and may be found in computerized databases or in the scientific literature, which is known to those of ordinary skill in the art. One such database is the National Center for Biotechnology Information's Genbank and GenPept databases (www.ncbi.nlm.nih.gov). All or part of the coding regions for these known genes may be amplified and/or expressed using the techniques disclosed herein or as would be know to those of ordinary skill in the art. Techniques inlcude PCR and other cellular, nucleic acid and protein manipulation methods.

In certain embodiments, a proteinaceous compound may be purified. Generally, “purified” will refer to a specific dsMHC I or dsMHC II polypeptide composition that has been subjected to fractionation to remove various other components including other proteins, polypeptides, or peptides, and which composition substantially retains its activity, as may be assessed, for example, by the protein, activity or binding assays described herein or known to one of ordinary skill in the art.

dsMHC I or dsMHC II polypeptides suitable for use in this invention may present allogeneic peptides. As used herein, the term “allogeneic polypeptide or peptide” refers to a protein, polypeptide or peptide which is derived or obtained from a genetically different organism of the same species. Organisms that may be used include, but are not limited to, bovine, rodent, avian, canine, or feline, with humans being preferred. The “allogeneic peptide” may then be used as a component of a composition intended for application to the selected animal or human subject, including samples derived therefrom. In certain aspects, an allogeneic peptide is derived from a cell, a tissue or an organ of a selected donor.

For diagnostic applications, the dsMHC I or dsMHC II polypeptides of the invention typically will be labeled with a detectable moiety. The detectable moiety can be any one which is capable of producing, either directly or indirectly, a detectable signal. For example, the detectable moiety may be a radioisotope, a fluorescent or chemiluminescent compound, such as fluorescein isothiocyanate, rhodamine, or luciferin; biotin; or an enzyme, such as alkaline phosphatase, beta-galactosidase or horseradish peroxidase.

Any method known in the art for separately conjugating a polypeptide to a detectable moiety may be employed, including those methods described by Hunter et al. (1962), David et al. (1974), Pain et al. (1981), and Nygren (1982).

The dsMHC I or dsMHC II polypeptides of the invention may be adapted and employed in any known antibody assay method, such as competitive binding assays, direct and indirect sandwich assays, and immunoprecipitation assays. Zola, 1987).

A. MHC Fusion Proteins

A specialized kind of insertional variant is the fusion protein. This molecule generally has all or a portion of the native molecule (e.g., a binding domain of an MHC I molecule), linked at the N- or C-terminus, to all or a portion of a second polypeptide (e.g., the constant region of an immunoglobulin molecule). For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as binding sites from cell surface molecules, dimerization domains, glycosylation domains, cellular targeting signals or transmembrane regions. Polypeptides, such as fusion protein, of the invention may be encoded by a nucleic acid that codes for such a polypeptide as described and exemplified herein.

In certain aspects of the invention, a nucleic acid encoding a dsMHC I or dsMHC II binding domain, in particular the alpha 1, alpha 2 and alpha 3 regions of an MHC I molecule or the extracellular domain of an MHC II molecule, is cloned. The nucleic acid encoding the binding domain may be derived from MHC I sequences known in the art and may include, but are not limited to (accession number/GI number) Human MHC class I HLA-A nucleotides (X13111/gi32138; X55710/gi32152; X60108/gi32157; M27539/gi187731; M24043/gi187775; M17690/gi188500; L18898/gi306853; U03754/gi432407; U07161/gi460241; D16841gi540516; D32129/gi699597; U50574/gi1245459; Z93949/gi1934950; U32184/gi2276447; AF015930/gi2323411; AJ011125/gi4128009; AB023056/gi5672625; AF217561/gi6815811; AJ278305/gi8250244; BC003069/gi13111763; BC008611/gi14250358; and BC019236/gi17512577), Human MHC class I HLA-B nucleotides (M16102/gi187693; M27540/gi187733; M59840/gi187758; M32317/gi187786; M24040/gi187807; M24032/gi187816; U04245/gi458663; X64454/gi474335; L33922/gi520834; U21052/gi695255; U21053/gi695257; X91749/gi1085023; U29057/gi1213466; U49905/gi1236148; U88407/gi3133270; Y13567/gi4007617; AF189017/gi6007812; AJ309047/gi13516327; AF436098/gi16903122; and AJ292075/gi21104319), and/or Human MHC class I HLA-C nucleotides (M11886/gi184173; and NM_{—}002117/gi19557676) and/or variants thereof. Furthermore, the nucleic acid encoding the binding domain may be derived from MHC II sequences known in the art and may include, but are not limited to (accession number/GI number) Human MHC class II HLA-DRB 1 nucleotides (X02902/gi30884; X03069/gi32283; M11161/gi188238; M33600/gi188240; M32578/gi188305; U65585/gi5478215; and AF029267/gi7643723); Human MHC class II HLA-DRA nucleotides (V00523/gi32125; J00194/gi188231; M60334/gi188255; K01171/gi188264; J00203/gi188426; J00204/gi188427; and BC032350/gi2161905), Human MHC class II HLA-DQB nucleotides (M65039/gi187942; K01499/gi187985; M60028/gi188114; M17955/gi188178; M17563/gi188182; M81140/gi188200; M81141/gi188202; M24364/gi529041; U92032/gi2665520; and BC012106/gi15082384); Human MHC class II HLA-DMA nucleotides (U04877/gi450815; U04878/gi450817; X76775/gi512468; BC011447 /gi15030335; BC026279/gi20072826); Human MHC class II HLA-DQA nucleotides (X00370/gi31762; X00452/gi32265; M33906/gi184194; M17846/gi187936; M17847/gi187938; M26041/gi188134; and BC008585/gi14250310); Human MHC class II HLA-DPB nucleotides (X03067/gi32275; X01426/gi36385; J03041/gi184192; M83664/gi188478; M28200/gi575493; M28202/gi575497;: BC007963/gi14044081; BC013184/gi15341974; and BC015000/gi15929087); Human MHC class II HLA-DRB5 nucleotides (M57648/gi187854; M20429/gi188394 M20430/gi188437; and BC009234/gi14328037); Human MHC class II HLA-DMB nucleotides (X76776/gi512471; U15085/gi557701; BC017508/gi19116258; BC027175/gi20073044; and BC035650/gi23272854)and/or variants thereof.

In certain embodiments, an dsMHC I or an dsMHC II binding domain may be fused to a dimerization domain. Dimerization domains may include, but are not limited to the C2 and C3 domains of an immunoglobulin molecule, in particular an IgG molecule. The dimerization domain may also include the hinge region of an immunoglobulin molecule. The nucleic acid encoding the dimerization domain may be derived from immunoglobulin sequences known in the art by standard molecular biology techniques and may include, but are not limited to the heavy chain of IgM (X17115/gi33450), IgG (J00228/gi184739), IgE (J00222/gi184755), and variants thereof.

The nucleic acid encoding the binding domain and the dimerization domain may be produced and manipulated using standard molecular biology techniques using the guidance provided herein to produce a fusion protein of the present invention.

B. Isolating dsMHC Polypeptides

Polypeptides of the invention may be obtained according to various standard methodologies that are known to those of skill in the art. For example, antibodies or other binding proteins specific for the polypeptides of the invention may be used in affinity protocols to isolate the respective polypeptide from cells, cell supernatants or cell lysates. Antibodies or other binding moieties can be advantageously bound to supports, such as columns or beads, and the immobilized antibodies or other binding moieties can be used to pull the dsMHC target out of the cell lysate or supernatant.

Expression vectors may be used to generate dsMHC polypeptides. A wide variety of expression vectors may be used, including viral vectors. The structure and use of these vectors is discussed further, below. Such vectors may significantly increase the amount of dsMHC protein produced by the cells, and may permit less selective purification methods such as size fractionation (chromatography, centrifugation), ion exchange or affinity chromatograph, and even gel purification.

It is expected that changes may be made in the sequence of a dsMHC polypeptide while retaining a molecule having the structure and function of a dsMHC polypeptide. For example, certain amino acids may be substituted for other amino acids in a protein structure without appreciable loss of interactive capacity with structures such as, for example, receptor-binding regions or T cells. These changes are termed “conservative” in the sense that they preserve the structural and, presumably, required functional qualities of the starting molecule.

C. dsMHC Polypeptide Variants

Conservative amino acid substitutions generally are based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as equivalent in certain circumstances.

In making such changes, the hydropathic index of amino acids also may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

In making changes based upon similar hydrophilicity values, the substitution of amino acids whose hydrophilicity values are within ±2 is preferred, those which are within ±1 are particularly preferred, and those within ±0.5 are even more particularly preferred.

Two designations for amino acids are used interchangeably throughout this application, as is common practice in the art. Alanine=Ala (A); Arginine=Arg (R); Aspartate=Asp (D); Asparagine=Asn (N); Cysteine=Cys (C); Glutamate=Glu (E); Glutamine=Gln (Q); Glycine=Gly (G); Histidine=His (H); Isoleucine=Ile (I); Leucine=Leu (L); Lysine=Lys (K); Methionine=Met (M); Phenylalanine=Phe (F); Proline=Pro (P); Serine=Ser (S); Threonine=Thr (T); Tryptophan=Trp (W); Tyrosine=Tyr (Y); Valine=Val (V).

D. Polypeptide Conjugates

Polypeptide conjugates comprising a dsMHC I or dsMHC II molecule linked to another agent including, but not limited to a therapeutic agent, a detectable label, a cytotoxic agent, a chemical, a toxin, an enzyme inhibitor, a pharmaceutical agent or an immunosupressant form further aspects of the invention. Diagnostic or detectable dsMHC conjugates may be used both in in vitro diagnostics, as in a variety of immunohistochemical assays, and in in vivo diagnostics, flow cytometry, and in vivo diagnostics such as in imaging technology.

Certain dsMHC conjugates include those intended primarily for use in vitro, where the dsMHC is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art and is described, for example, in U.S. Pat. Nos. 3,817,837; 3,850,752; 3,939,350; 3,996,345; 4,277,437; 4,275,149 and 4,366,241; each incorporated herein by reference.

In using a dsMHC-based molecule as an in vitro or in vivo diagnostic agent to provide an image of, for example, biopsies, blood, brain, thyroid, breast, gastric, colon, pancreas, renal, kidney, ovarian, lung, cardiac, hepatic, lung tissues or samples thereof, by immunohistochemistry, magnetic resonance imaging, X-ray imaging, computerized emission tomography and other similar technologies may be employed. In the dsMHC-imaging compositions of the invention, the dsMHC I OR dsMHC II portion used will generally bind to markers for alloreactivity, such as alloreactive T cells, and the imaging agent will be an agent detectable upon imaging, such as a paramagnetic, radioactive, immunohistochemical or fluorescent agent.

Many appropriate imaging agents are known in the art, as are methods for their attachment to polypeptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to an antibody (U.S. Pat. No. 4,472,509, which is incorporated herein by reference).

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, carbon¹⁴, chromium⁵¹, chlorine³⁶, cobalt⁵⁷, cobalt⁵⁸, copper⁶⁷, Eu¹⁵², gallium⁶⁷, hydrogen³, iodine¹²³, iodine , iodine¹³¹, indium¹¹¹, iron⁵⁹, phosphorus³², rhenium¹⁸⁶, rhenium¹⁸⁸, selenium⁷⁵, sulphur³⁵, technicium^{99m} and yttrium⁹⁰.

Radioactively labeled dsMHC I or dsMHC II of the present invention may be produced according to well-known methods in the art. For instance, iodination by contact with sodium or potassium iodide and a chemical oxidizing agent such as sodium hypochlorite, or an enzymatic oxidizing agent, such as lactoperoxidase. dsMHC I or dsMHC II according to the invention may be labeled with technetium-^{99m} by ligand exchange process, for example, by direct labeling techniques, e.g., by incubating pertechnate, a reducing agent such as SNCl₂, a buffer solution such as sodium-potassium phthalate solution, and the dsMHC.

Intermediary functional groups which are often used to bind radioisotopes which exist as metallic ions to polypeptides are diethylenetriaminepentaacetic acid (DTPA) and ethylene diaminetetracetic acid (EDTA). Fluorescent labels include rhodamine, fluorescein isothiocyanate and renographin.

E. Expression Systems

Systems for expressing polypeptides of the invention include prokaryote- and/or eukaryote-based systems. These systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

In certain embodiments, polypeptides of the invention may be transcribed, translated, processed and/or secreted by a producer cell, i.e. a cell used for the purpose of producing a particular polypeptide or polypeptide complex, e.g., dsMHC I/β2 molecular complex.

III. Nucleic Acids

Certain embodiments of the present invention concern a nucleic acid sequence encoding a dsMHC I or dsMHC II. In particular aspects, a nucleic acid encodes for or comprises a transcribed nucleic acid.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, a uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

In certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this functional term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express or may be adapted to express various proteins, polypeptides, domains, peptides, fusion proteins, or mutant polypeptides of the invention.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

“Isolated substantially away from other coding sequences” means that the gene of interest forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria-(see for example, Sambrook et al. 2001, incorporated herein by reference).

B. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of the bulk of the total genomic and transcribed nucleic acids of one or more cells. In certain embodiments, “isolated nucleic acid” refers to a nucleic acid that has been isolated free of the bulk of cellular components or in vitro reaction components, such as, macromolecules including lipids, proteins, small biological molecules, and the like.

C. Nucleic Acid Segments

In certain embodiments, the nucleic acid is a nucleic acid segment. As used herein, the term “nucleic acid segment,” are smaller fragments of a nucleic acid, a non-limiting example including those that encode only part of dsMHC I or dsMHC II polypeptide. Thus, a “nucleic acid segment” may comprise any part of a gene sequence, of from about 8 nucleotides to a full length dsMHC I or ds MHC II.

Various nucleic acid segments may be designed based on a particular nucleic acid sequence, and may be of any length. Exemplary nucleic acid sequences of dsMHC molecules include, but are not limited to SEQ ID NO: 1 and SEQ ID NO:3. By assigning numeric values to a sequence, for example, the first residue is 1, the second residue is 2, etc., an algorithm defining all nucleic acid segments can be created:
n to n+y
where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm may be applied to each of the sequences described herein. As used herein, a “probe” generally refers to a nucleic acid used in a detection method or composition. As used herein, a “primer” generally refers to a nucleic acid used in an extension or amplification method or composition.

In a non-limiting example, one or more nucleic acid constructs may be prepared that include a contiguous stretch of nucleotides identical to or complementary to the referenced sequences. A nucleic acid construct may be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 60, about 70, about 80, about 90, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 14, about 15, about 16, about 17, about 18, about 19, about, 20, about 21, about 22, about 23, about 24, about 25, about 26, about 27, about 28, about 29, about 30, about 35, about 40, about 50, about 60, about 70, about 80, about 90, about 100, about 125, about 150, about 175, about 200, about 500, about 1,000, about 10,000, about 50,000, about 100,000 or more bases.

D. Nucleic Acid Complements

The present invention also encompasses a nucleic acid that is complementary to a referenced sequence. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence, e.g., SEQ ID NO:1 or SEQ ID NO:3, is capable of base-pairing with a single or double stranded nucleic acid molecule of dsMHC I or dsMHC II during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

E. Genetic Degeneracy

The term “functionally equivalent codon” is used herein to refer to codons that encode the same amino acid, such as the six codons for arginine and serine, and also refers to codons that encode biologically equivalent amino acids. For optimization of expression in human cells, the codons are shown in Table 1 in preference of use from left to right. Thus, the most preferred codon for alanine is thus “GCC”, and the least is “GCG” (see Table 1 below). Codon usage for various organisms and organelles can be found at the website www.kazusa.orjp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

TABLE 1
Preferred Human DNA Codons

| | |
|---|---|
| Amino Acids | Codons |

| | | | | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gin | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of a dsMCH I or dsMHC II will be nucleic acid sequences that are “essentially dsMHC I or dsMHC II sequences.”

F. dsMHC Vectors and Expression Constructs

In various embodiments, polypeptides of the invention, e.g., dsMHC I or dsMHC II molecules, may be expressed in vitro, ex vivo and/or in vivo. Nucleic acids encoding these polypeptides may be comprised in vectors or expression vectors. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell where it is ordinarily not found. Vectors include plasmids, cosmids, viruses (bacteriophage, animal viruses, and plant viruses), and artificial chromosomes (e.g., YACs). One of skill in the art would be well equipped to construct a dsMHC vector through standard recombinant techniques (see, for example, Sambrook et al., 2001 and Ausubel et al., 1996, both incorporated herein by reference).

The term “expression vector” refers to any type of genetic construct comprising a nucleic acid coding for a RNA capable of being transcribed. In some cases, RNA molecules are then translated into a polypeptide. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well and are described infra.

In order to express an ds MHC polypeptide it is necessary to provide an dsMHC I or dsMHC II gene in an expression vehicle or cassette. The appropriate nucleic acid can be inserted into an expression vector by standard subcloning techniques. For example, an E. coli or baculovirus expression vector is used to produce recombinant polypeptide in vitro. The manipulation of these vectors is well known in the art. In one embodiment, the protein is expressed as a fusion protein with a peptide tag, allowing rapid affinity purification of the protein. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these fusion systems produce recombinant protein bearing only a small number of additional amino acids, which are unlikely to affect the functional capacity of the recombinant protein. For example, both the FLAG system and the 6×His system add only short sequences, both of which are known to be poorly antigenic and which do not adversely affect folding of the protein to its native conformation. Other fusion systems produce proteins where it is desirable to excise the fusion partner from the desired protein. In another embodiment, the fusion partner is linked to the recombinant protein by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

There are a variety of eukaryotic vectors that provide a suitable vehicle in which recombinant ds MHC polypeptide can be produced. HSV has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Another preferred embodiment is the tetracycline controlled promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 2 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally, any promoter/enhancer combination (as per the Eukaryotic Promoter Data Base EPDB) could also be used to drive expression of a transgene. Eukaryotic cells can support cytoplasmic transcription from certain bacterial promoters if the appropriate bacterial polymerase is provided, either as part of the delivery complex or as an additional genetic expression construct. Promoters of the invention include, but are not limited to lmmunoglobulin Heavy Chain, Immunoglobulin Light Chain, T-Cell Receptor, HLA DQ α and DQ β, β-Interferon, Interleukin-2, Interleukin-2 Receptor, MHC Class II 5, MHC Class II HLA-DRα, β-Actin, Muscle Creatine Kinase, Prealbumin (Transthyretin), Elastase I, Metallothionein, Collagenase, Albumin Gene, α-Fetoprotein, τ-Globin, β-Globin, c-fos, c-HA-ras, Insulin, Neural Cell Adhesion Molecule (NCAM), α_{1-Antitypsin}, H2B (TH2B) Histone, Mouse or Type I Collagen, Glucose-Regulated Proteins (GRP94 and GRP78), Rat Growth Hormone, Human Serum Amyloid A (SAA), Troponin I (TN I), Platelet-Derived Growth Factor, Duchenne Muscular Dystrophy, SV40, Polyoma, Retroviruses, Papilloma Virus, Hepatitis B Virus, Human Immunodeficiency Virus, Cytomegalovirus, or Gibbon Ape Leukemia Virus promoters.

TABLE 2

| Element | Inducer |
|---|---|
| MT II | Phorbol Ester (TPA) |
| | Heavy metals |
| MMTV (mouse mammary tumor virus) | Glucocorticoids |
| β-Interferon | Poly(rI)X |
| | Poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle |
| | Disease Virus |
| GRP78 Gene | A23187 |
| □-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone □ | Thyroid Hormone |
| Gene | |

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

1. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of nucleic acids into cells (e.g., mammalian cells). Vector components of the present invention may be a viral vector that encode one or more ds MHC I, dsMHC II or other components such as, for example, an immunomodulator. Non-limiting examples of virus vectors that may be used to deliver a nucleic acid of the present invention include adenoviral vectors (Grunhaus and Horwitz, 1992), AAV vectors (Kelleher and Vos, 1994; Cotten et al., 1992; Curiel, 1994 and U.S. Pat. Nos. 5,139,941 and 4,797,368, each incorporated herein by reference), retroviral vectors (Miller, 1992), and lentiviral vectors (Naldini et al., 1996; Zufferey et al., 1997; Blomer et al., 1997; U.S. Pat. Nos. 6,013,516 and 5,994,136). Other viral vectors may be employed such as vaccinia virus (Ridgeway, 1988; Baichwal and Sugden, 1986; Coupar et al., 1988), sindbis virus, cytomegalovirus and herpes simplex virus. They offer several attractive features for various mammalian cells (Friedmann, 1989; Ridgeway, 1988; Baichwal and Sugden, 1986; Coupar et al., 1988; Horwich et al., 1990).

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

G. Vector Delivery and Cell Transformation

Suitable methods for nucleic acid delivery for transformation of a cell, a tissue or an organism for use with the current invention are believed to include virtually any method by which a nucleic acid (e.g., DNA) can be introduced into a cell, a

tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042; 5,322,783, 5,563,055, 5,550,318, 5,538,877 and 5,538,880, and each incorporated herein by reference); and any combination of such methods. Through the application of techniques such as these, cell(s), tissue(s) or organism(s) may be stably or transiently transformed. Stably transfected cells are preferred in embodiments that utilize a cell expressing dsMHC I or dsMHC II as a therapeutic.

H. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector or an expression cassette. A host cell can, and has been, used as a recipient for vectors. A host cell may be “transfected” or “transformed,” by exogenous nucleic acid that is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors, e.g., a dsMHC I and a β2m vector. Alternatively, a single recombinant vector may be constructed to include multiple distinct coding regions for RNAs, which could then be expressed in host cells transfected with the single vector.

In certain embodiments, the host cell or tissue may be comprised in at least one organism. In certain embodiments, the organism may be, but is not limited to, a prokaryote (e.g., a eubacteria, an archaea) or an eukaryote, as would be understood by one of ordinary skill in the art (see, for example, phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org), as well as being isolated from all or part of a donor organism. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5a, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, primary cells derived from a graft donor, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using all or part of a dsMHC I or dsMHC II sequence(s).

IV. Therapeutics, T Cell Detection, and Diagnostics

Proteinaceous and/or cellular compositions of the invention may be used therapeutically to inhibit, modulate or abrogate immune responses, in particular alloreactive immune responses. For example, immune responses that may be modulated include the ability to depress or abrogate T-cell proliferation, T- and B-cell growth and differentiation, acute phase reaction, IL-3 and IL-4 involvement in hematopoiesis, cytokine activation or inhibition, IL2 and IFN gamma involvement in inflammation, T-cell responses, inflammation, apoptosis and calcium-independent cytotoxicity.

A transplantation antigen is a molecule responsible for graft recognition. Because the immunological status of the recipient is a critical factor affecting graft survival, diverse antigen systems may be involved in the acceptance/rejection process. These systems not only include the well recognized HLA system, i.e., class I and class II MHC molecules, but also include other minor histocompatibility antigens, such as the ABO blood group system, (including carbohydrates, which includes but is not limited to, disaccharides, trisaccharides, tetrasaccharides, pentasaccharides, oligosaccharides, polysaccharides, and more preferably, the carbohydrate α (1,3) galactosyl epitope [α (1,3) Gal]), autoantigens on T and B cells, and monocyte/endothelial cell antigens. Since the present invention is primarily concerned with dsMHC compositions comprising two MHC binding domains, transplantation antigens in the context of the present invention include MHC class I or class II antigens. In clinical applications concerning treatment or therapy to detect, inhibit, reduce or abrogate T cell alloreactivity, selective suppression of antigen specific responses are targeted. A transplantation antigen may be any class I or class II MHC molecule, or more specifically for humans, any MHC molecules including HLA specificities such as A (e.g., A1-A74), B (e.g., B1-B77), and C (e.g., C1-C11). More preferably, serological HLA specificities include A1, A2, A3, A11, A23, A24, A28, A30, A33, B7, B8, B35, B44 B53, B60, B62, D or variants thereof (Zachary et al., 1996).

A patient who has received or will receive an organ transplant can be treated with molecular complexes or a cell expressing molecular complexes, proteinaceous or cellular compositions of the invention. Therapeutic applications involve the specific suppression of alloreactivity to a transplantation antigen using dsMHC I or dsMHC II molecules of the present invention.

dsMHCs or cells expressing dsMHCs in which each MHCs binding domain is bound to an antigent or alloantigen can be administered to a patient at a dose sufficient to suppress, reduce or abrogate an immune response to the cell, tissue or organ transplant.

A patient who suffers from an allograft rejection can be treated with molecular complexes or compositions of the invention in which the binding domain is associated with an antigenic peptide to which the patient expresses an alloreactive response. The compositions are administered to the patient at a dose sufficient to suppress, reduce or abrogate the immune response.

Because the expression constructs of the present invention can incorporate a signal sequence for the secretion of each fusion protein, it is possible that the therapeutic methods of the present invention may also be performed with polynucleotides or vectors designed for gene therapy. The polynucleotide may be DNA or RNA. When the polynucleotide is DNA, it can also be a DNA sequence which is itself non-replicating, but is inserted into a replicating plasmid vector. The polynucleotide may be engineered such that it is or is not integrated into the host cell genome. Alternatively, the polynucleotide may be engineered for integration into the chromosome in which the expression may or may not be controlled. Regulatable gene expression systems having in vivo applicability are known in the art, and may be used in the present invention.

A. Pharmaceutical Formulations

The compositions of the present invention can be provided in unit dosage form, wherein each dosage unit, e.g., a teaspoon, tablet, or solution, contains a predetermined amount of the composition, alone or in appropriate combination with other pharmaceutically-active agents. The term “unit dosage form” refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically-acceptable diluent, carrier (e.g., liquid carrier such as a saline solution, a buffer solution, or other physiological aqueous solution), or vehicle, where appropriate. The specifications for the novel unit dosage forms of the present invention depend on the particular effect to be achieved and the particular pharmacodynamics associated with the pharmaceutical composition in the particular host.

Compositions comprising or expressing proteinaceous compositions of the invention can comprise a pharmaceutically acceptable carrier or diluent. Pharmaceutically acceptable carriers and diluents which are soluble in the circulatory system and which are physiologically acceptable are well known to those in the art. “Physiologically acceptable” means that those skilled in the art would accept injection of said carrier into a patient as part of a therapeutic regime. The carrier preferably is relatively stable in the circulatory system or body cavities. Suitable carriers include, but are not limited to, water, alcoholic/aqueous solutions, emulsions, or suspensions, including saline and buffered media, and proteins such as serum albumin, heparin, immunoglobulin, polymers such as polyethylene glycol or polyoxyethylated polyols or proteins modified to reduce antigenicity by, for example, derivitizing with polyethylene glycol. Suitable carriers are well known in the art and are described, for example, in U.S. Pat. No. 4,745,180, 4,766,106, and 4,847,325, which are incorporated herein by reference. Pharmaceutically acceptable carriers also include, but are not limited to, large, slowly metabolized macromolecules, such as proteins, polysaccharides, polylactic acids, polyglycolic acids, polymeric amino acids, amino acid copolymers, and inactive virus particles. Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Compositions of the invention can also contain liquids, such as water, saline, glycerol, and ethanol, as well as substances such as wetting agents, emulsifying agents, or pH buffering agents. Liposomes, such as those described in U.S. Pat. No. 5,422,120, PCT applications WO 95/13796 and WO 91/14445, or European Patent EP 524,968 B1, can also be used as a carrier for a composition of the invention.

If appropriate, pharmaceutical compositions may be formulated into preparations including, but not limited to, solid, semi-solid, liquid, or gaseous forms, such as tablets, capsules, powders, granules, ointments, solutions, injections, inhalants, and aerosols, in the usual ways for their respective route of administration. Methods known in the art can be utilized to prevent release or absorption of the composition until it reaches the target organ or to ensure time-release of the composition. A pharmaceutically-acceptable form should be employed which does not inactivate or render the compositions of the present invention ineffective.

Other medications or compounds may be adminstered in conjunction with the compositions of the invention including prophylactic agents such anti-virals, anti-bacterials and other anti-microbials that may reduce the likelihood of an infection(s).

In pharmaceutical dosage forms, the compositions may be used alone or in appropriate association, as well as in combination with, other pharmaceutically-active compounds. For example, in applying the method of the present invention for delivery of dsMHCs of the invention, which contain an antigen binding domain or region of an MHC I molecule that dimerize forming a functional unit involved in immune modulation, such delivery may be employed in conjunction with other means of treatment of alloreactive immunity. The compounds of the present invention may be administered or expressed alone or in combination with other diagnostic, therapeutic or additional agents.

Therapeutic agents may include immunosuppressants such as cyclopsorin, rapamycin and other immunosupressants known to one of ordinary skill in the art. The dsMHCs of the invention may be administered or expressed with or without supplementation with an immunosuppressant, e.g., cyclosporine A for the induction of a permanent allograft. Other immunosuppressants include, but are not limited to rapamycin, azathiprine, cyclophosphamide, mycophenolate, daclizumab, prednisone, muromonab, sirolimus, tacrolimus, and steroids. Various other immunosuppressants can be identified in the Physicians' Desk Reference (PDR), 57^{th} edition, Published by Medical Economics; (November 2002), which is incorporated herein by reference.

Additionally, the present invention specifically provides a method of administering soluble constructs of the invention to a host, which comprises administering a composition of the present invention using any of the aforementioned routes of administration or alternative routes known to those skilled in the art and appropriate for the particular application. In certain embodiments, a cell expressing dsMHC I dsMHC II molecule(s) may be administered to a subject. The cell may or may not engraft into a tissue or organ of the subject. The engrafted cell would provide a continuous administration of dsMHC I or dsMHC II to a subject.

The particular dosages of dsMHC molecular complexes employed for a particular method of treatment will vary according to the condition being treated, the binding affinity of the particular reagent for its target, the extent of disease progression, etc. However, the dosage of molecular complexes will generally fall in the range of 1 pg/kg to 100 mg/kg of body weight per day. Where the active ingredient of the pharmaceutical composition is a polynucleotide encoding fusion proteins of a molecular complex, dosage will generally range from 1 nM to 50 μM per kg of body weight.

The amounts of each active agent included in the compositions employed in the examples described herein provide general guidance of the range of each exemplary component that may be utilized by the practitioner upon optimization of the method. Moreover, such ranges by no means preclude use of a higher or lower amount of a component, as might be warranted in a particular application. For example, the actual dose and schedule may vary depending on whether the compositions are administered in combination with other pharmaceutical compositions, or depending on individual differences in pharmacokinetics, drug disposition, and metabolism. Similarly, amounts may vary for in vitro applications depending on the particular cell line utilized, e.g., the ability of the plasmid employed to replicate in that cell line. For example, the amount of nucleic acid to be added per cell or treatment will likely vary with the length and stability of the nucleic acid, as well as the nature of the sequence, and may be altered due to factors not inherent to the method of the present invention, e.g., the cost associated with synthesis, for instance. One skilled in the art can easily make any necessary adjustments in accordance with the necessities of the particular situation.

Accordingly, the pharmaceutical compositions of the present invention can be delivered via various routes and to various sites in an animal body to achieve a particular effect. Local or systemic delivery can be accomplished by administration comprising application or instillation of the formulation into body cavities, inhalation, or inhalation of an aerosol, or by parenteral introduction, comprising intramuscular, intravenous, peritoneal, subcutaneous intradermal, as well as topical administration.

A sufficient dose of the composition for a particular use is that which will produce the desired effect in a host. This effect can be monitored using several end-points known to those skilled in the art. For example, one desired effect might comprise effective nucleic acid transfer to a host cell. Such transfer could be monitored in terms of a therapeutic effect, e.g., alleviation of some alloreactivity associated with immune response being treated, or further evidence of the transferred gene or expression of the gene within the host, e.g., using PCR, Northern or Southern hybridization techniques, or transcription assays to detect the nucleic acid in host cells, or using immunoblot analysis, antibody-mediated detection, or the assays described in the examples below, to detect protein or polypeptide encoded by the transferred nucleic acid, or impacted level or function due to such transfer.

In other embodiments, in addition to the therapies described above, one may also provide to the patient more “standard” therapies. Examples of standard therapies include, without limitation, anti-microbials, anti-virals, antibiotics, hormones, and steroids.

Combinations may be achieved by administering to an organism a single composition or pharmacological formulation that includes both agents, or by administering to an organism two distinct compositions or formulations, at the same time, wherein one composition includes an expression construct or dsMHC and the other includes the second agent, e.g. immunosuppressant or anti-microbial. Alternatively, gene or cellular therapy may precede or follow administration of the other agent by intervals ranging from minutes to weeks. In embodiments where the other agent and an expression construct, cellular therapy or dsMHCs are applied separately to the cell, one would generally ensure that a significant period of time did not expire between the time of each delivery, such that the agent and dsMHC therapy, either proteinaceous, cellular or a gene therapy, would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a dsMHC therapy, or the other agent will be desired. In this regard, various combinations may be employed. By way of illustration, where dsMHC therapy is “A” and the other agent or immunosuppressant is “B,” the following permutations based on 3 and 4 total administrations are exemplary:

A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B

Other combinations are likewise contemplated.

B. Detection and Diagnostics

Compositions comprising dsMHCs of the invention can be used diagnostically, to label or detect T cells in vitro or in vivo. A sample comprising T cells, preferably alloreactive T cells, can be contacted with a dsMHC I or dsMHC II. The sample can be, for example, peripheral blood, lymphatic fluid, lymph nodes, spleen, thymus, bone marrow, cerebrospinal fluid, [or biopsy or histological sectio of allograft}.

The dsMHC can specifically bind to an alloreactive T cell and label it with the dsMHC. dsMHCs can be, but need not be, conjugated to a reporter group, such as a radiolabel (e.g., ³²P) or fluorescent label, an enzyme, a substrate, a solid matrix, or a carrier (e.g., biotin or avidin) to facilitate detection of specific molecules or the binding activity of a dsMHC molecules of the present invention. The dsMHC polypeptide(s) can be in solution or can be directly or indirectly affixed to a solid substrate, such as a glass or plastic slide or tissue culture plate or latex, polyvinylchloride, or polystyrene beads. Constructs of the present invention may be further modified to include toxins or other therapeutic molecules. In certain embodiments the dsMHC may be detected by contacting a sample that has been contacted with dsMHC with one or more additional detection agents, such as an antibody, colorimetric or fluorescent reagents, or combinations thereof. Various secondary detection methods are known in the art and are exemplified herein.

T cells that bind to dsMHC polypeptides can be separated from other cells that are not bound. Any method known in the art can be used to achieve this separation, including plasmapheresis, flow cytometry, or differential centrifugation. T cells of a patient or isolated from a patient can be contacted with a composition comprising dsMHC polypeptide(s) to provide a prophylactic or therapeutic effect. Optionally, the number of T cells that are bound to or by the dsMHC polypeptide can be quantitated or counted, for example by flow cytometry or microscope.

A sample which comprises alloreactive T cells can be contacted, in vivo or in vitro, with dsMHC molecules in which each antigen binding site is bound to an antigenic peptide, preferably an alloreactive antigen.

V. dsMHC Kit

For use in the methods described above, kits are also provided by the invention. Such kits may include any or all of the following: dsMHC (conjugated or unconjugated, or cell expressing a dsMHC); a pharmaceutically acceptable carrier (may be pre-mixed with the dsMHC) or suspension base for reconstituting lyophilized dsMHC; additional medicaments; a sterile vial for each dsMHC and additional medicament, or a single vial for mixtures thereof; device(s) for use in delivering dsMHC to a host; assay reagents for detecting indicia that the anti-inflammatory and/or immunosuppressive effects sought have been achieved in a treated cell or subject, and a suitable assay device.

Certain embodiments include a detection kit according to the present invention comprising all of the essential reagents required to perform a desired assays according to the present invention for the detection of alloreactive T cells in a test sample. The test kit is presented in a commercially packaged form as a combination of one or more containers holding the necessary reagents, as a composition or admixture where the compatibility of the reagents will allow.

Particularly preferred is a test kit for a fluorescent (or other detectable moiety) assay for detection of alloreactive T cells in a test sample, comprising fluorescent compounds conjugated to dsMHC molecules or other detection molecules as described hereinabove. It is to be understood that the test kit can, of course, include other materials as are known in the art and which may be desirable from a user standpoint, such as buffers, diluents, standards, and the like, useful as washing, processing and indicator reagents.

Examples illustrating the practice of the invention are set forth below. The examples are for purposes of reference only and should not be construed to limit the invention, which is defined by the appended claims. All abbreviations and terms used in the examples have their expected and ordinary meaning unless otherwise specified.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Methods

Antibodies and Rats

The antibodies anti-CD8α, anti-CD4, OX18 (anti RT1.A, polymorphic), anti-rat IgG_{2c}, and anti-mouse IgG alkaline phosphatase-conjugated antibody were purchased from Pharmingen, USA. The peroxidase conjugated rabbit anti-mouse IgG was obtained from Jackson ImmunoResearch Laboratories, USA. The rat IgG_{2c} control immunoglobulin was obtained from Serotec, UK. The anti-FLAG antibody was purchased from Sigma, USA. Eight to twelve-week-old rats were purchased from Jackson Laboratories, USA. The DA (RT1.A^{a}, Lewis (RT1.A¹, and BN (RT1.A^{u}) rats were purchased and maintained at the VA Medical Center Iowa City Animal Care Center.

Construction and Expression of Dimeric RT1.A¹-Fc

The full length cDNA of RT1.A¹ was kindly provided by Dr. T. J. Gill III and Dr. S. K. Salgar (Genbank-Accession no: L26224, the sequence of which is incorporated herein by reference), Pittsburgh, Pa., USA. The first four exons of RT1.A¹ were amplified using polymerase chain reaction (PCR). The full length construct served as a template. During amplification, a SmaI restriction site was attached to the 5′-end and a XbaI restriction site was added at the 3′-end. Generated PCR fragments were ligated into the SmaI/XbaI-linearized eukaryotic expression vector pRK5 (Pharmingen, San Diego, Calif., USA). In addition, DNA encoding for the hinge-, C2- and C3-region of a rat IgG_{2c} molecule (Genbank-Accession no: X07189, the sequence of which is incorporated herein by reference) was amplified from spleen cDNA and the resulting PCR-fragment (bp 292-990) was ligated into the TA-cloning site of the pCR2.1 vector (Invitrogen, Carlsbad, USA). During amplification, a XbaI restriction site was attached to the 5′-end and a HindIII restriction site was added at the 3′-end. Subsequently, the XbaI/HindIII IgG-fragment was subcloned into the RT1.A¹/pRK5 vector and DNA encoding for a FLAG-tag was added by PCR at the 3′-end. The resulting SmaI/HindIII DNA-fragment (1638 bp), was thereafter subcloned into the expression vector pcDNA3.1- (Invitrogen, Carlsbad, USA) and was used for generating stable transfectants expressing recombinant dimeric RT1.A¹ molecules (RT1.A¹). Each primer set was designed with regard to the full-length sequences published in Genbank and synthesized by Life Technologies (Berlin, FRG). The generated rsRT1.A¹ and IgG_{2c} sequences were confirmed by DNA-sequencing using the ABI373 DNA-Sequencer (Perkin-Elmer, Norwalk, Conn., USA).

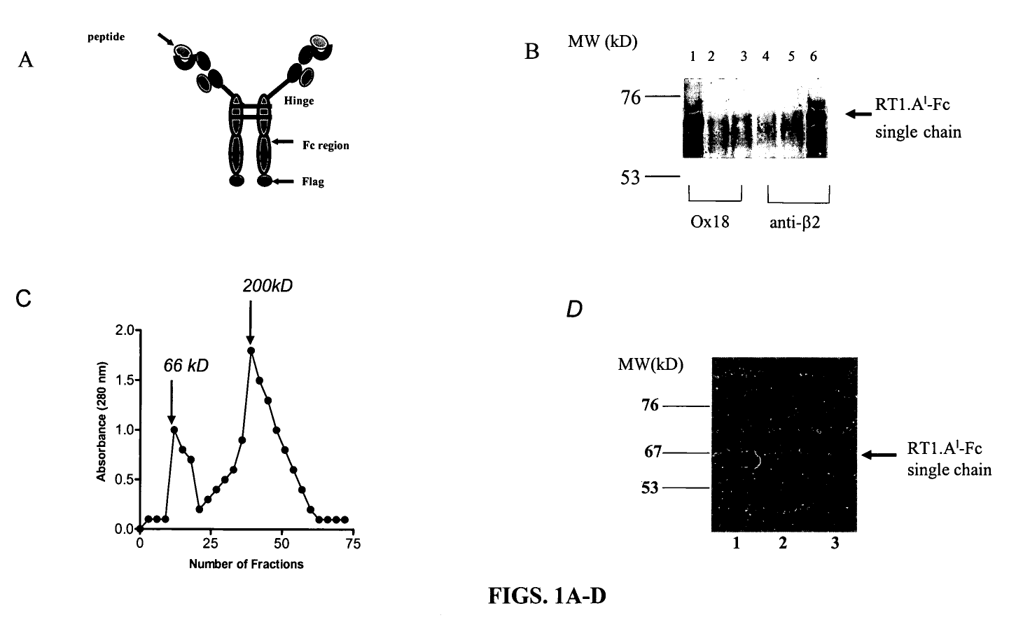

The rat myeloma cell line Y3-Ag 1.2.3 (ATCC, Rockville, Md., USA) was chosen for recombinant expression of dimeric RT1.A¹-Fc because it does not express an IgG heavy chain but β2-microglobulin. Cells were transfected using effectene (Qiagen, Hilden, FRG) according to the manufacturer's instructions. Briefly, 10⁶ cells were seeded in DMEM-Medium (ICN, USA) on 100-mm dishes (Falcon, 3803) 18 h prior to transfection. After 18 h, culture-medium was changed. Ten μg DNA were incubated with EC-buffer, enhancer and effectene at a ratio of 1:5 and supplemented with medium to each dish. Transfected cells were selected in DMEM medium containing 800 μg/ml G418 (Calbiochem, San Diego, Calif.) for at least 5 weeks. Cell clones were generated under selecting conditions by limiting dilution. The schematic structure of the constructed MHC class I dimer is shown in FIG. 1A and its predicted molecular mass is 170 kD.

Quantitative Enzyme-Linked Immunosorbent Assay (ELISA)

A monoclonal rat anti-IgG2c (Pharmingen, USA) antibody was coated on one half of a 96-well micro-titer ELISA plate (Nunc, Denmark) at a concentration of 1 μg/ml and left to incubate overnight at 37° C. in a humidified chamber. Similarly, an anti-FLAG monoclonal antibody (MoAb, Sigma,USA) was also pre-coated on the other half of the plate at a concentration of 14 μg/ml. The plate was washed 3 times with PBS/0.1%Tween 20 and subsequently blocked using 5% milk powder supplemented with 2% mouse normal serum in PBS. As standard antigen, a serial dilution of a rat IgG2c (Serotec, UK) was used within the concentration range of 1-10 ng/ml and added to the wells pre-coated with the anti-rat IgG2c antibody, whereas transfectant supernatant containing RT1.A¹-Fc were added to the other half of the plate pre-coated with an anti-FLAG antibody and incubated for 90 minutes. The plates were washed 5 times and biotinylated anti IgG2c (1 μg/ml) was added in all the wells and the plate was incubated for another 45 minutes. After washing 5 times, a streptavidin-horseraddish-peroxidase conjugate was added to each well and incubated for 45 minutes. The plates were washed 9 times and peroxidase substrate solution added. The reaction was stopped using 2N HCl. Absorbance was read at 492 nm on a Biomek 1000 (Beckman). The concentration of the RT1.A¹-Fc was determined using the standard IgG2c.

Immunoprecipitation and Western Blotting of Recombinant RT1.A¹-Fc

One milliliter culture supernatant of transfected cells grown in serum-free medium was incubated with shaking at 4° C. with either 2 μg of a polyclonal anti-β-2 microglobulin serum (Dako, Danemark) or 2 μg of OX-18, a monoclonal antibody against a conformational-dependent epitope on heavy chains of rat MHC class I molecules. After 4 hours, 50 μl of protein-A agarose (Pharmacia, Sweden) were added to each sample and the mixture was shaken overnight at 4° C. Samples were washed thoroughly in PBS and centrifuged. The pellets were resuspended in 100 μl SDS-loading buffer, boiled and centrifuged. The recovered supernantant was run on a 7.5% polyacrylamide gel and blotted on a nitrocellulose membrane. The proteins were stained using an anti-FLAG monoclonal antibody. Protein bands were visualized by an anti-mouse IgG alkaline phosphatase-conjugated antibody.

Transplantation of Cardiac Allografts

To test the efficacy of the soluble dimer to promote graft survival, DA recipient animals received 10 μg of the dimer by intraperitoneal infusion daily for 14 days. They were transplanted with a cardiac allograft on the first day according to Ono and Lindsey (1969) and either left untreated (n=6) or received a single dose of 15 mg/kg CsA (n=12). Graft function was monitored by palpation. Lewis-derived cardiac allografts were transplanted to DA recipients as previously reported (Fandrich et al., 1999). In this strain combination cardiac allografts are acutely rejected on day 7.

Immuno-Histochemical and Flow Cytometric Staining

Cryostat sections recovered on day 5 (rejecting animals) or on day 150 (tolerant animals) were fixed in methanol, washed in Tris-HCl (pH 7.6) and incubated with the dimer. After further washing, dimer binding was visualized by the use of a goat anti-mouse serum conjugated with alkaline phosphatase. To detect alloreactive T cells by flow cytometry, 25 μl of peripheral blood were incubated with a lysis solution containing ammonium chloride to remove the erythrocytes. The peripheral blood lymphocytes or splenocytes were incubated with the dimer for 30 minutes on ice. The cells were washed and then incubated with a FITC-conjugated anti-FLAG monoclonal antibody. After another 30 minutes, the cells were again washed and finally incubated with a PE-conjugated anti-CD8 monoclonal antibody. The cells were then washed and cell fluorescence measured in a FACScan (BDPharmingen).

Immunomodulation of Alloreactive T Cells by Intraperitoneal Injection of RT1.A¹-Fc

Proliferation assays—Here, the inventors tested the efficacy of injecting alloantigen beginning with the day of immunization with donor-derived splenocytes. Thus, DA recipient animals were immunized daily from day 0 to day 14 with the dimeric MHC antigen. Each dose contained approximately 10 μg/ml. 107 Lewis-derived splenocytes were infused on day 0 and repeated on day 7 to sensitize the host T cells. Animals were sacrificed on day 14 and spleens harvested. Splenocytes were used either directly in proliferation assays or stimulated in vitro for another 5 days using irradiated Lewis-derived splenocytes at a 1:1 ratio.

Cytotoxicity assay—To test whether donor-derived dimeric alloantigen modulates T cell cytotoxicity, the bulk cultures generated above were used as effector cells. Target cells were either the Y3-Ag1.2.3 cells transfected with the Lewis-derived membrane bound RT1.A¹ or Lewis-derived Con A blast cells. T cell cytotoxicity was tested in a 4 h chromium release assay (Behrens et al., 2001). The inhibition assay of alloreactive T cells was performed as previously described (Freese and Zavazava, 2002; Zavazava et al., 1991; Charlton and Zmijewski, 1970).

Measurement of IFN-γ production by ELISPOT—To measure the secretion of IFN-γ in the treated groups of animals, splenocytes were derived from animals sensitized with splenocytes only, with splenocytes and treated with the dimer or from non-treated control animals, which were used as responder cells to irradiated Lewis-derived splenocytes. IFN-γ secretion was measured by the use of an IFN-γ ELISPOT kit (BDPharmingen). Spots were counted using an automated ELISPOT reader system (Immunospot, Cellular Technology Ltd., USA).

### Example 2

Results

Characterization of Dimeric RT1.A¹-Fc

Transfected cells secreted the dimer in serum-free medium as determined by a newly established conformational-dependent quantitative ELISA. Transfectants were cloned by limiting dilution and clones secreting >15 μg/ml of the dimer were further maintained in culture. To determine the molecular size of this molecule, the dimer was immunoprecipitated using OX18 and the anti-β2-microglobulin serum, respectively. A major band of 67 kD was detected in samples precipitated with either the OX18 or with the anti-132-microglobulin antibody, FIG. 1B. In supernatants derived from some clones, an additional minor band slightly less than the major 67 kD band was detected. This protein was precipitated by both antibodies suggesting that it is a spliced product of the full length dimeric MHC. Thus, both the OX-18 MoAb (anti-RT1.A conformationally folded heavy chain) and the anti-β2 microglobulin polyclonal serum were able to recover the recombinant dimeric MHC-class I molecule indicating that the recombinant class I dimer was properly folded and heterodimeric, containing both the class I heavy chain and the β2-microglobulin. To confirm that the recombinant protein was dimeric, the dimer was purified by affinity chromatography using a protein A column, and run on a G200 gel filtration column. Since the transfected cells were grown in serum-free medium, only 2 major protein peaks of 66 and 200 kD were observed. As tested by ELISA, most of the recombinant material was eluted in the 200 kD fraction, FIG. 1C, which matches the predicted 170 kD protein size of the dimer. The purified dimer was run on western blots after gel filtration and detected by the anti-FLAG antibody. As expected, a single band of 67 kD representing the single chain of the dimer was detected, FIG. 1D.

Dimeric RT1.A¹-Fc Induce Indefinite Graft Survival of Allogeneic Cardiac Allografts

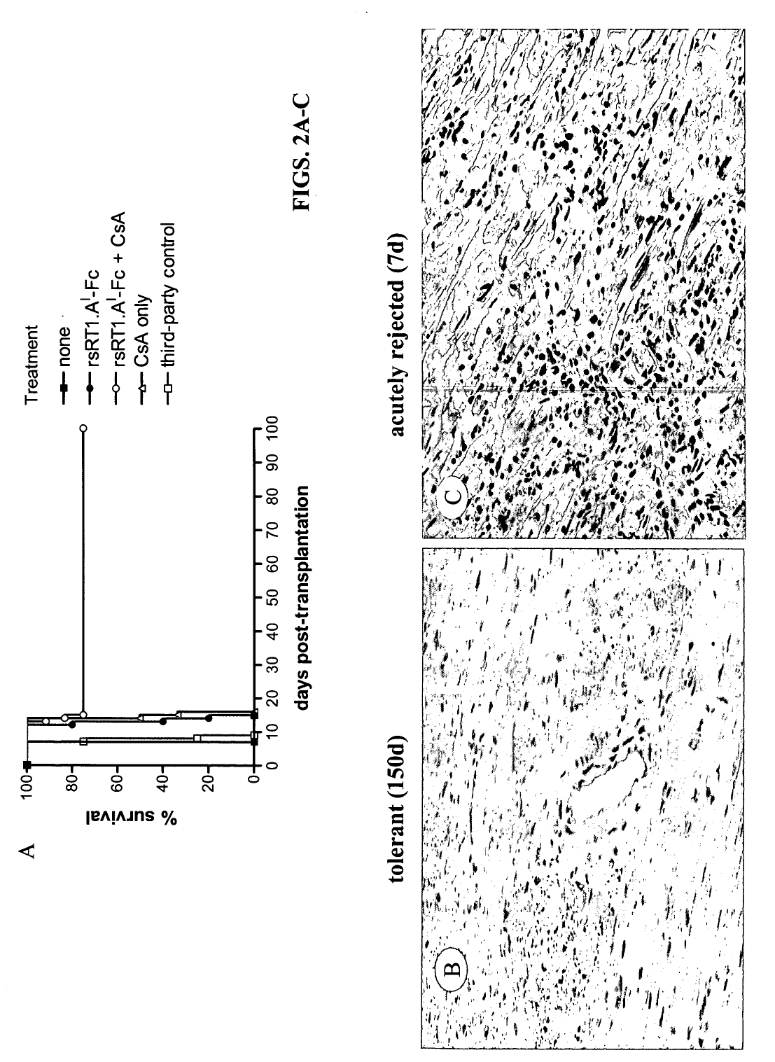

DA recipient animals were infused daily 10 μg of the RT1.A¹-Fc for 14 days. This protocol was a modification of our previous protocol using monomeric sMHC (Behrens et al., 2001). Together with the first dimer infusion, recipient animals were transplanted Lewis-derived heterotopic cardiac transplants and either left untreated or received a single dose of 15 mg/kg cyclosporine A. Treatment of recipient animals with this dose of cyclosporine A alone prolonged graft survival from 7 days to 14 days. Surprisingly, the dimeric MHC antigen equally prolonged graft survival, 8.5±0.1 to 13.2±0.4 days (p value <0.0001; n=10) indicating the efficacy of the molecules to abrogate graft rejection. In contrast, 9 out of a total of 12 animals or 75 % treated with the dimer and subtherapeutic cyclosporine A showed indefinite graft survival, >150 days post-transplantation, FIG. 2A. Third party BN-derived cardiac allografts were rejected by day 9. These results are highly superior to published findings with synthetic MHC peptides or with monomeric soluble MHC antigens (Behrens et al., 2001; Freese and Zavazava, 1996), where only 50% prolonged graft survival was achievable.

However, prolonged graft survival is not always accompanied by lack of pathology in the allograft. For example, vascular occlusion as a consequence of chronic rejection has been reported in allografts despite prolonged graft survival. Therefore, animals were sacrificed 150 days post-transplantation and histological sections stained and analyzed. There was no graft infiltration characteristic of rejection and more importantly, no signs of intimal thickening as a sign of chronic rejection, FIGS. 2B-2C. Multiple sections were studied to detect any signs of rejection. Interestingly, graft infiltration on day 7 in the group that received the dimer was similar to that after 150 days, showing that the dimer had an impact on the homing of T cells in this model. In contrast, acutely rejected allografts showed heavy infiltration of myocardial tissue by mononuclear cells. Tissue damage was extensive and the muscle architecture of the allografts obliterated, clearly indicating severe acute rejection.

Treatment with Dimeric RT1.A1-Fc Modulates T Cell Responses to Alloantigen

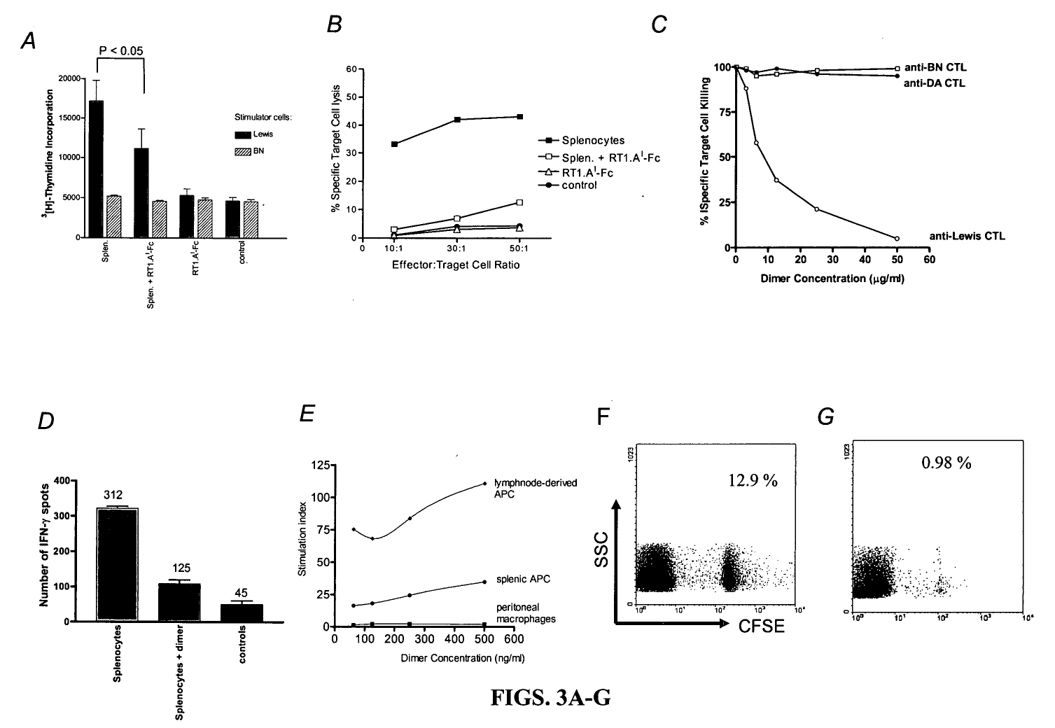

To understand the mechanism by which donor-derived dimeric MHC protected allografts from rejection, recipient animals were treated with the dimeric MHC antigen for 14 days as described above. Together with the first dimer infusion, animals were immunized with 10×10⁶ donor-derived splenocytes. This infusion was repeated on day 7 and the animals sacrificed on day 14. Spleens were harvested and their response to alloantigen analyzed. First, T cells were used as responder cells in a 4-day mixed-lymphocyte assay. Control groups were animals treated with the dimer only, splenocytes only or untreated animals, respectively. As expected, CD8+ T cells recovered from animals sensitized with splenocytes only showed strong proliferation to donor-type alloantigen, FIG. 3A. Surprisingly, proliferation of T cells recovered from animals that were treated with the dimer in addition to the splenocytes was significantly abrogated, p<0.05. The other two control groups showed limited T cell proliferation. However, alloresponse to third-party responder animals of the BN strain, remained unchanged in all groups. A similar response was observed in CD4+ T cells (data not shown), suggesting that the dimer was downregulating both CD4+ and CD8+ T cells. As expected, low level CsA (1 μg/ml) blocked proliferation of both CD4- and CD8-positive T cells.

To investigate the effect of the treatment protocol on cytotoxic T cells against the donor strain, splenocytes harvested from treated animals were restimulated in vitro using irradiated Lewis-derived donor cells for 5 days. CD8+ T cells were isolated by immunomagnetic beads and tested for their cytotoxicity against donor derived Con A blast cells. The highest cytotoxicity was observed with cells derived from the animals sensitized with donor-type splenocytes only, FIG. 3B. However, cytotoxicity was abrogated by pre-treatment with the dimer despite sensitization with donor splenocytes. The Y3-Ag 1.2.3 cells transfected with membrane bound RT1.A1 or Lewis-derived Con A blast cells were used as target cells. The previous studies had shown that alloreactive T cells were blocked by monomeric soluble MHC and by their synthetic peptides (Freese and Zavazava, 2002; Zavazava and Kronke, 1996). Here, the inventors tested whether the dimeric MHC class I molecule abrogated alloreactive CTL. Indeed, DA-derived anti-LewisCTL were blocked by the dimer in a concentration-dependent manner as determined in a 4 h-51chromium-release assay. However, syngeneic anti-DA CTL or DA-derived anti-BN CTL were not affected by the dimer in their ability to recognize alloantigen on target cells, FIG. 3C. To determine the effect of the dimer on CD4+ alloreactive T cells, DA responder rats were sensitized with Lewis-derived splenocytes only or with the splenocytes and dimer over 14 days. The animals were sacrificed and CD4+ T cells isolated and used to determine their ability to produce IFN-γ after alloantigen stimulation. Indeed the cells derived from animals treated with the dimer and splenocytes showed significant reduction in IFN-γ production after alloantigen stimulation, FIG. 3D, as compared to the cells derived from animals sensitized with the splenocytes only. These results altogether indicated that dimer treatment effectively modulated both CD4+ and CD8+ T cells.

To explain these unexpected sMHC-induced effects, it was hypothesized that peritoneal macrophages are involved in the presentation of the dimer, leading to downregulation of alloreactive T cells. CD80 and CD86 were measured on the DA-derived peritoneal macrophages after a peritoneal lavage and were found to be 1.7 and 2.1% respectively, as compared to 8.1 and 33.3% in splenocytes, suggesting that peritoneal macrophages were likely to provide weak co-stimulation leading to T cell anergy rather than stimulation. Indeed, when the peritoneal macrophages were used as stimulator cells in a proliferation assay with the dimer used as alloantigen, they failed to activate alloreactive T cells, FIG. 3E. To determine whether peritoneal macrophages trafficked to peripheral lymphoid organs after picking up alloantigen, the inventors harvested peritoneal macrophages and pulsed them with the dimer over 24 h ex vivo. The cells were washed and subsequently labeled with CFSE, re-infused into the peritoneum of DA recipient animals and sacrificed 24 h later to determine the presence of fluorescent green cells in the peritoneum, peripheral blood, spleens and lymph-nodes. About 1% of circulating leukocytes were CFSE labeled compared to 12.9% in the peritoneal lavage (FIGS. 3F and 3G). Less than 0.5% of splenocytes or lymphnode-derived cells were CFSE positive. These results suggested that peritoneal macrophages trafficked into the circulation and into peripheral lymphoid organs after engagement with alloantigen, where they likely interacted with T cells.

Dimeric MHC Molecules Visualize Alloreactive T Cells

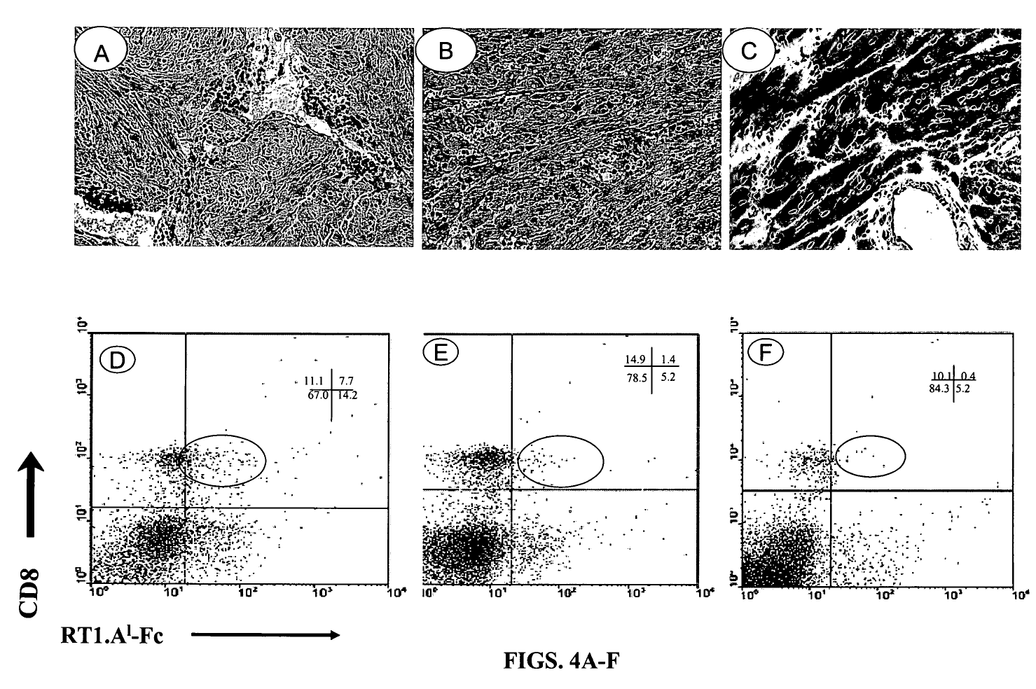

The inventors further investigated whether alloreactive T cells can be visualized by immunohistochemical staining using the dimer. Only a few cells were stained by the dimer on the tolerated allograft (FIG. 4A). These cells were confirmed to be CD8+ by double-staining (not shown). As expected, parenchymal tissue of the tolerated allografts was intact. In contrast, rejected allografts showed a diffuse to dense distribution of mononuclear cells most of which were positive for the dimer, FIG. 4B. Our stains indicated that CD8+ cells in rejected allografts were about 50-60% of the total mononuclear cell population detected by immunohistochemistry. Control sections from animals transplanted with BN third party allografts showed negligible dimer staining, FIG. 4C. Thus, these data indicated that the dimer can be used for staining alloreactive T cells in allografts and that the frequency of alloreactive CD8+ cells was high within the rejected allografts.

In order to validate these findings, peripheral blood was drawn from DA rats rejecting Lewis-derived cardiac allografts on day 7 post-transplantation, from tolerant animals (day 60) or from non-transplanted control animals. Full blood was double-stained with the dimer and a fluorescein-conjugated anti-CD8 antibody. Representative stains are shown in FIGS. 4D-4F. In each group, 6 animals were tested. Approximately 2.0±1.8% of the cells were double positive for CD8 and the dimer on tolerated allografts, FIG. 4E, as compared to 6.2±1.2% detected in peripheral blood of rejecting animals, FIG. 4D. T lymphocytes derived from control animals showed no dimer binding, FIG. 4F. These data indicated that about 3 times more alloreactive T cells were stained by the dimer in rejecting animals than in tolerant animals suggesting that the dimer could be a novel tool for monitoring alloresponses using whole blood as a source of alloreactive T cells.

The MHC Dimer Detects a High Frequency of Alloreactive CD8+ T Cells in Splenocytes of Rejecting Animals

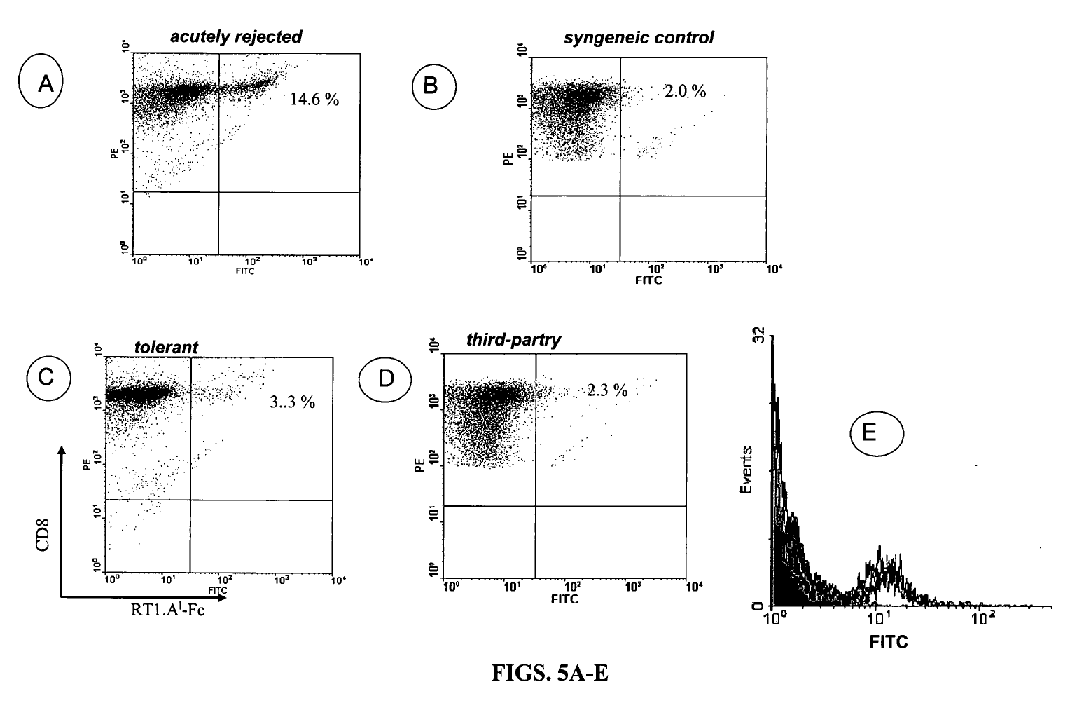

To further characterize alloreactive T cells using the dimeric MHC molecules, Lewis-derived cardiac allografts were transplanted into DA recipient animals and left untreated. The organs were rejected on day 7 and the animals sacrificed for the recovery of spleens. CD8+ T cells were separated from splenocytes by immunomagnetic beads and stained with the dimer. Clearly, 14.6% CD8 high of the splenocytes in rejecting animals were positive for the dimer compared to only 3.3% in tolerant animals (n=6) sacrificed on day 40 post transplantation, FIG. 5. To further confirm the specificity of this stain, splenocytes recovered from DA animals rejecting DA allografts (syngeneic control) and from third-party controls (BN transplanted in Lewis) were used as controls (FIG. 5). In both control groups, only 2% of the cells were stained. Since the dimer has an IgG backbone and can potentially bind to the FcR on T cells, splenocytes were pre-incubated from animals rejecting a Lewis allograft with antibodies against CD4, CD8, FcR or CD28, respectively. After 1 h incubation, the cells were washed to remove excess antibody and then stained with the dimer to detect any changes in the amount of dimer binding. None of these antibodies interferred with the dimer staining, clearly showing that the stains obtained were not due to the dimer binding to the cells via the FcR or any of the tested molecules, FIG. 5E. Further, dimer-binding cells were isolated by immunomagnetic separation and used as responder cells in a proliferation assay. Control cells were CD8+ cells obtained from a control animal. Indeed, dimer binding cells from a rejecting animal responded to alloantigen 3-4 times more strongly than non-dimer binding cells (data not shown), clearly confirming that the cells binding the dimer were alloreactive. Collectively, the data presented here further confirmed that the dimeric MHC molecule indeed visualizes alloreactive T cells in allospecific fashion.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 4,659,774
U.S. Pat. No. 4,682,195
U.S. Pat. No. 4,683,202
U.S. Pat. No. 4,745,180
U.S. Pat. No. 4,766,106
U.S. Pat. No. 4,797,368
U.S. Pat. No. 4,816,571
U.S. Pat. No. 4,847,325
U.S. Pat. No. 4,879,236
U.S. Pat. No. 4,959,463
U.S. Pat. No. 5,021,236
U.S. Pat. No. 5,139,941
U.S. Pat. No. 5,141,813
U.S. Pat. No. 5,264,566
U.S. Pat. No. 5,322,783
U.S. Pat. No. 5,384,253
U.S. Pat. No. 5,422,120
U.S. Pat. No. 5,428,148
U.S. Pat. No. 5,434,131
U.S. Pat. No. 5,538,877
U.S. Pat. No. 5,538,880
U.S. Pat. No. 5,550,318
U.S. Pat. No. 5,554,744
U.S. Pat. No. 5,563,055
U.S. Pat. No. 5,574,146
U.S. Pat. No. 5,580,859
U.S. Pat. No. 5,589,466
U.S. Pat. No. 5,602,244
U.S. Pat. No. 5,610,042
U.S. Pat. No. 5,645,897
U.S. Pat. No. 5,656,610
U.S. Pat. No. 5,702,932
U.S. Pat. No. 5,705,629
U.S. Pat. No. 5,736,524
U.S. Pat. No. 5,780,448
U.S. Pat. No. 5,789,215
U.S. Pat. No. 5,871,986
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 6,458,354
Arimilli et al., Immunol. Cell Biol., 74(1):96-104, 1996.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Behrens et al., Transplantation, 72:1974-1982, 2001.
Bittner et al., Methods in Enzymol, 153:516-544, 1987.
Blomer et al., J. Virol., 71(9):6641-6649, 1997.
Bodmer et al. In: Immunobiology of HLA, Dupont (Ed.), Vol 1, New York: Springer-Verlag, 1989.
Bryn et al., Nature, 344:667-670, 1990.
Capon et al., Nature, 337:525-531, 1989.
Casares, et al., Nat. Immunol., 3:383-391, 2002.
Charlton and Zmijewski, Science, 170(958):636-637, 1970.
Chen and Okayama, Mol. Cell Biol., 7(8):2745-2752, 1987.
Corr et al., Science, 265:946-949, 1994.
Cotten et al., Proc. Natl. Acad. Sci. USA, 89(13):6094-6098, 1992.
Coupar et al., Gene, 68:1-10, 1988.
Curiel, Nat. Immun., 13(2-3):141-164, 1994.
Dal Porto et al., Proc. Natl. Acad. Sci. USA, 90(14):6671-6675, 1993.
David et al., Biochemistry, 13:1014, 1974.
EP 266 032
EP 524,968 B1
Fandrich et al., J. Leukoc. Biol., 65:16-27, 1999.
Fechheimer et al., Proc. Natl. Acad. Sci. USA, 84:8463-8467, 1987.
Fraley et al., Proc. Natl. Acad. Sci. USA, 76:3348-3352, 1979.
Freese and Zavazava, Blood, 99(9):3286-32892, 2002.
Freese and Zavazava, Transpl. Int., 9(1):S352-355 1996.
Friedmann, Science, 244:1275-1281, 1989.
Froehler et al., Nucleic Acids Res., 14(13):5399-5407, 1986.
Ghio et al., Blood, 93:1770-1777, 2000.
Gill et al., Immunol. Rev., 149:75-96, 1996.
Gopal, Mol. Cell Biol., 5:1188-1190, 1985.
Graham and van der Eb, Virology, 52:456-467, 1973.
Grunhaus et al., Seminar in Virology, 200(2):535-546, 1992.
Haga et al., J. Biol. Chem., 266:3695-3701, 1991.
Hansen et al., Transplantation, 66:1818-1822, 1998.
Harland and Weintraub, J. Cell Biol., 101(3):1094-1099, 1984.
Hausmann et al., Clin. Exp. Immunol., 91:183-188, 1993.
Hebell et al., Science, 254:102-105, 1991.
Herz and Roizman, Cell, 33(1):145-151, 1983.
Horwich et al. J. Virol., 64:642-650, 1990.
Hunter et al., Nature 144:945 (1962)
June et al., Immunol. Today, 15 (7):321-331, 1994.
Kaneda et al., Science, 243:375-378, 1989.
Kao et al., Human Immunol., 21:115-124, 1988.
Kato et al, J. Biol. Chem., 266:3361-3364, 1991.
Kelleher and Vos, Biotechniques, 17(6):1110-7, 1994.
Kyte and Doolittle, J. Mol. Biol., 57(1):105-32, 1982.
Lenschow et al., Science, 257:789-791, 1992.
Linsley et al., Science, 257:7920-795, 1992.
Matsui et al., Science, 254:1788-1891, 1991.
Miller et al., Am. J. Clin. Oncol., 15(3):216-221, 1992.
Nabel et al., Science, 244(4910):1342-1344, 1989.
Nag et al., J. Biol. Chem.,271(17):10413-10418, 1996.
Naldini et al., Science, 272(5259):263-267, 1996.
Nicolau and Sene, Biochim. Biophys. Acta, 721:185-190, 1982.
Nicolau et al., Methods Enzymol., 149:157-176, 1987.
Nygren, Histochem. and Cytochem., 30:407, 1982.
O'Herrin et al., J. Immunol., 167:2555-2560, 2001.
Ono and Lindsey, J. Thorac. Cardiovasc. Surg., 57:225-229, 1969.
Pain et al., J. Immunol. Meth., 40:219, 1981.
Paul, In: Fundamental Immunology, 3^{RD} Ed., Raven Press, New York, 1993.
PCT Appln. WO 91/14445
PCT Appln. WO 94/09699
PCT Appln. WO 95/06128
PCT Appln. WO 95/13796
Physicians'Desk Reference (PDR), 57th edition, Published by Medical Economics, 2002.
Potter et al., Proc. Natl. Acad. Sci. USA, 81:7161-7165, 1984.
Pouletty et al., Tissue Antigens, 42:14-19, 1993.
Priestley et al., Transplantation, 48:1031-1038, 1989.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Roux et al., Proc. Natl. Acad. Sci. USA, 86:9079-9083, 1989.
Sambrook et al., In: Molecular cloning, Cold Spring Harbor Laboratory Press, Cold Spring Harbor, N.Y., 2001.
Schneck et al., Cell, 56:47-55, 1989.
Schneck et al., Hum. Immunol., 47:149, 1996.
Schneck et al., Proc. Natl. Acad. Sci., 86:8516-8520, 1989.
Schodin et al., Immunity, 5:137-146, 1996.
Shoskes and Wood, Immunology Today, 15(1):32-38, 1994.
Sykulev et al., Immunity, 1: 15-22, 1994.
Tur-Kaspa et al., Mol. Cell Biol., 6:716-718, 1986.
Wilson et al., Science, 244:1344-1346, 1989.
Wong et al., Gene, 10:87-94, 1980.
Wu and Wu, Biochemistry, 27:887-892, 1988.
Wu and Wu, J. Biol. Chem., 262:4429-4432, 1987.
Zachary et al., Transplant, 62, 272-283, 1996.
Zavazava and Kronke, Nat. Med., 2(9):1005-1010, 1996.
Zavazava et al., J. Immunogenetics, 17:387-394, 1990.
Zavazava et al., Tissue Antigens, 42:20-26, 1993.
Zavazava et al., Transplantation, 51:838-842, 1991.
Zola, In: Monoclonal Antibodies: A Manual of Techniques, 147-158, CRC Press, Inc., 1987.
Zinkemagel et al., Nature, 248:701-702, 1974.
Zufferey et al., Nat. Biotechnol., 15(9):871-875, 1997.

1. A polypeptide comprising MHC class I alpha 1, alpha 2 and alpha 3 domains operatively coupled to a dimerization domain, wherein the polypeptide lacks an immunoglobulin variable region domain.

2. The polypeptide of claim 1, wherein the dimerization domain is an immunoglobulin C2-C3 domain.

3. The polypeptide of claim 1, further comprising an amino terminal leader sequence.

4. The polypeptide of claim 3, wherein the leader sequence is a MHC class I leader sequence.

5. The polypeptide of claim 1, further comprising a carboxy-terminal peptide tag.

6. The polypeptide of claim 5, wherein the peptide tag is a Flag tag.

7. A polynucleotide comprising a nucleic acid sequence that encodes a polypeptide comprising MHC class I alpha 1, alpha 2 and alpha 3 domains fused with a carboxy-terminal dimerization domain, wherein the polypeptide lacks an immunoglobulin variable region.

8. The polynucleotide of claim 7, further comprised in an expression cassette comprising a promoter.

9. The polynucleotide of claim 8, further comprised in an expression vector.

10. The polynucleotide of claim 9, wherein the expression vector is a eukaryotic expression vector.

11. The polynucleotide of claim 10, wherein the eukaryotic expression vector is a viral expression vector.

12. The polynucleotide of claim 7, further comprising a nucleic acid sequence encoding a carboxy-terminal peptide tag.

13. The polynucleotide of claim 7, further comprising a nucleic acid sequence encoding an amino terminal leader sequence.

14. A proteinaceous composition comprising a homodimeric molecule, wherein each monomer comprises MHC class I alpha 1, alpha 2 and alpha 3 domains operatively coupled to a dimerization domain, wherein each monomer lacks an immunoglobulin variable domain.

15. The proteinaceous composition of claim 14, wherein the dimerization domain is an immunoglobulin C2-C3 domain.

16. The proteinaceous composition of claim 14, wherein each monomer comprises an amino terminal leader sequence.

17. The proteinaceous composition of claim 16, wherein the leader sequence is a MHC class I leader sequence.

18. The proteinaceous composition of claim 14, wherein at least one monomer comprises a peptide tag.

19. The proteinaceous composition of claim 18, wherein the peptide tag is a Flag tag.

20. The proteinaceous composition of claim 14, wherein composition is a pharmaceutically acceptable composition.

21. A proteinaceous composition comprising a dimeric complex of polypeptides, wherein a first and second polypeptide comprise MHC I antigen binding region operatively coupled to at least one immunoglobulin constant region, wherein the first and second polypeptides each lack immunoglobulin variable regions.

22. A cell comprising a nucleic acid coding for a polypeptide comprising a MHC I antigen binding region operatively coupled to a dimerization domain, wherein the polypeptide lacks an immunoglobulin variable region domains.

23. A method of producing a dimeric polypeptide molecule comprising:
a) contacting a host cell with a nucleic acid encoding a polypeptide comprising MHC class I alpha 1, alpha 2 and alpha 3 domains operatively coupled to a dimerization domain, wherein the polypeptide lacks an immunoglobulin variable region domains; and
b) isolating a dimeric polypeptide molecule.

24. A method comprising contacting an alloreactive T-cell with an effective amount of a dimeric molecule comprising a first and second polypeptide comprising MHC I or MHC II antigen binding region operatively coupled to at least one immunoglobulin constant region, wherein the first and second polypeptides each lack an immunoglobulin variable region.

25. The method of claim 24, wherein the T-cell is in a subject.

26. The method of claim 24, wherein the T-cell is in a tissue transplant.

27. The method of claim 26, wherein the tissue is an organ.

28. The method of claim 25, further comprising administering to the subject an effective dose of an immunosuppressant.

29. The method of claim 24, wherein the dimeric molecule is administered by intravascular or intra peritoneal injection or infusion.

30. The method of claim 28, wherein the immunosuppressant is cyclosporin.

31. The method of claim 24, wherein an effective amount of the dimeric molecule is at a dose of about 1 μg to 100 μg per kg of body weight.

32. The method of claim 31, wherein the dose is about 10 μg to 50 μg per kg of body weight.

33. A method comprising:
a) contacting a sample suspected of containing alloreactive T-cells with an effective amount of a dimeric molecule comprising a first and second polypeptide comprising MHC I or MHC II antigen binding region operatively coupled to at least one immunoglobulin constant region, wherein the first and second polypeptides each lack an immunoglobulin variable region; and
b) visualizing a T-cell by contacting the T-cell/dimeric molecule complex with a detection reagent.

34. The method of claim 33, wherein the sample is peripheral blood, splenocytes, or all or part of a transplanted tissue.

35. A kit comprising a first container comprising a proteinaceous composition comprising dimeric complex of polypeptides, wherein a first and second polypeptide comprise a MHC I or MHC II antigen binding region operatively coupled to at least one immunoglobulin constant region, wherein the first and second polypeptides each lack an immunoglobulin variable region.

36. The kit of claim 35, further comprising a second container comprising a reagent for detection of the dimeric polypeptide.
